SEQUENCE LISTING (7 sequences)

SEQ ID NO: 1 - protein, 121 aa, synthetic construct
MSEIDVEIVAVEREIWSGKATFVFTRTTSGEIGILPHHIPLVAQLVDDAAVKIEREGSDDLWWAIDGGFLSITDTKVSILAESAQARADIDEAKAKTDSGSEDPRVAAQGRARLRALGQTV

SEQ ID NO: 2 - protein, 308 aa, synthetic construct
MGAQIRELRRRIKSAGAIKKITKAQELIATSRIAKAQARVVAARPYATEITNVLTALADDAALDHPLLVERPEPKRAGVLIVSSDRGLCGGYNANVLRVAEELYALLREQGKTPVVYVVGRKALNYYSFRNRKVTEAWTGFSERPEYASAQKIADTLVEAFLAGADDEGDDPGLDGILGVDELHIVYTEFKSMLTQAAVAKRIAPMEVEYVGEAAGPTTQYSFEPDATTLFGALLPRYLATRVYAALLEAAASESASRRRAMKAATDNADELIKGLTLEANGARQAQITQEISEIVGGVNALADAAGH

SEQ ID NO: 3 - protein, 168 aa, synthetic construct
MLPAALLRRPGLGRLVRHARAYAEAAAAPAAASGPNQMSFTFASPTQVFFNGANVRQVDVPTLTGAFGILAAHVPTLQVLRPGLVVVHAEDGTTSKYFVSSGSIAVNADSSVQLLAEEAVTLDMLDLGAAKANLEKAQAELVGTADEATRAEIQIRIEANEALVKALE

SEQ ID NO: 4 - protein, 139 aa, synthetic construct
MAMTYHLDVVSAEQQMFSGLVEKIQVTGSEGELGIYPGHAPLLTAIKPGMIRIVKQHGHEEFIYLSGGILEVQPGNVTVLADTAIRGQDLDEARAMEAKRKAEEHISSSHGDVDYAQASAELAKAIAQLRVIELTKKAM

SEQ ID NO: 5 - protein, 121 aa, synthetic construct; identical to SEQ ID NO: 1

SEQ ID NO: 6 - protein, 121 aa, synthetic construct
MSEIDVEIVAVEREIWSGKATFVFTRTTSGEIGILPHHIPLVAQLVDDAAVKIEREGSDDLWWAIDGGFLSITDTKVSILAESAQARADIDESKAKTDSGSEDPRVAAQGRARLRALGQTV

SEQ ID NO: 7 - protein, 121 aa, synthetic construct; identical to SEQ ID NO: 6

(19) United States
(12) Patent Application Publication
(10) Pub. No.: US 2024/0390351 A1
(43) Pub. Date: Nov. 28, 2024

(54) MYCOBACTERIUM ABSCESSUS GROWTH INHIBITOR AND MODEL

(71) Applicant: Nanyang Technological University, Singapore (SG)

(72) Inventors: Gerhard GRÜBER, Singapore (SG); Joon SHIN, Singapore (SG); Venkata Krishna Harikishore AMARAVADHI, Singapore (SG); Chui Fann WONG, Singapore (SG); Priya RAGUNATHAN, Singapore (SG)

(21) Appl. No.: 18/691,686

(22) PCT Filed: Sep. 21, 2022
(86) PCT No.: PCT/SG2022/050676

§ 371 Date: Mar. 13, 2024

(30) Foreign Application Priority Data
Sep. 21, 2021 (SG) 10202110405X

(51) Int. Cl. A61K 31/4439; A61P 31/04; C07D 401/12; G16B 15/30; G16B 35/00

(52) U.S. Cl. CPC A61K 31/4439; A61P 31/04; C07D 401/12; G16B 15/30; G16B 35/00

(57) ABSTRACT

This invention relates to compounds having the generic structure of formula I: X-L-Y—Z I which are useful in the treatment of a bacterial infection caused by Mycobacterium abscessus, where: X is selected from an unsubstituted or substituted phenyl, pyridyl, quinazolinyl, or naphtyl; Y is an unsubstituted or substituted heteroaryl ring system selected from benzimidazolyl, benzothiazolyl, benzofuranyl, quinazolinyl, and naphthyl; Z is selected from H, phenyl and pyridyl, where the phenyl and pyridyl groups are unsubstituted or substituted; L is selected from —CH2NHCOCH2CH2- or —CH2-phenyl-CH2, —CH2-, —NH-pyrrolyl, imidazolyl, and thiazolyl or a pharmaceutically acceptable salt or solvate thereof.

### FIELD OF INVENTION

The current invention relates to growth inhibitors of Mycobacterium abscessus, as well as to a model system that may be useful for screening potential compounds.

### BACKGROUND

The listing or discussion of a prior-published document in this specification should not necessarily be taken as an acknowledgement that the document is part of the state of the art or is common general knowledge.

The incidence and prevalence of nontuberculous mycobacterial (NTM) infections have been increasing worldwide and present an emerging public health problem. Among rapidly growing NTM, Mycobacterium abscessus (Mab) is the most common and the most drug-resistant opportunistic pathogen. Mab has been classified into the three subspecies Mycobacterium abscessus subsp. abscessus, Mycobacterium abscessus subsp. massiliense, and Mycobacterium abscessus subsp. bolletii, with M. abscessus subsp. abscessus being the most prevalent and resistant one. Challenges in Mab treatment comprise long, non-standardized administration of antimicrobial agents with poor treatment outcomes, often related to drug toxicities, as well as high relapse rates. Most of the administered anti-Mab drugs require minimum inhibitory concentrations (MIC₅₀) for growth inhibition in a micromolar range. The problem of Mab treatment goes along with an anti-Mab drug pipeline that remains poorly populated, highlighting the need for novel targets and inhibitors.

The process of oxidative phosphorylation (OXPHOS) is the major process for Mab to synthesize the currency of life, ATP, making this pathway vulnerable to potential drugs. This is demonstrated by bedaquiline (BDQ) and its derivative TBAJ876, which are active against Mab by targeting the F₁F₀ ATP synthase. The mycobacterial enzyme (FIG. 1A) consists of an F₁ domain, including subunits α₃:β₃:γ:ε, the proton-translocating F₀ domain (subunits a:c₉), and the peripheral stalk subunits b-δ:b′. The F₁ domain contains three catalytic αβ-pairs that form an α₃:β₃ hexamer, in which ATP formation occurs. The rotational movement of the c-ring triggers the central subunits γ and ε to rotate, causing sequential conformational changes in the nucleotide-binding subunits α and β, followed by the synthesis of ATP. Besides the rotation movement of subunit ε, its interdomain conformational changes are proposed to transmit the power between the rotary c-ring and the α₃:β₃ domain, making it an essential coupling element of the mycobacterial F-ATP synthase engine. This was demonstrated for the M. smegmatis subunit ε (Msε) counterpart, where an E mutant showed decreased intracellular ATP, slower growth rates and lower molar growth yields on nonfermentable carbon sources. Furthermore, cellular respiration and metabolism were all accelerated in the mutant strain indicative of dysregulated oxidative phosphorylation. An amino acid sequence alignment of mycobacterial ε subunits in FIG. 1B highlights the differences in the three M. abscessus subspecies to their counterparts in other mycobacteria, which have been described to be critical in coupling and regulation. So far, no structural information of Mab's F-ATP synthase or any of its subunits has been described.

Therefore, there exists a need to identify the structural information of Mab's F-ATP synthase or any of its subunits and to discover new inhibitors of Mab.

### SUMMARY OF INVENTION

Aspects and embodiments of the invention are discussed by reference to the following numbered clauses.

1. A compound of formula I:

X-L-Y—Z I
where:
X is selected from phenyl, pyridyl, quinazolinyl, or naphtyl, where the phenyl, pyridyl, quinazolinyl, or naphtyl group is unsubstituted or substituted by one or more of the group selected from Cl, NH₂, pyrrolyl, imidazolyl, tetrazolyl, CH₂NHCONH₂, and CH₂NHSO₂NH₂;
Y is a heteroaryl ring system selected from benzimidazolyl, benzothiazolyl, benzofuranyl, quinazolinyl, and naphthyl, which groups are unsubstituted or substituted by one or more substituents selected from the group consisting of OMe, —O—CH₂—O—, and —O(CH₂)₂O—, where the oxygen atoms in the latter two groups are attached to different atoms on the heteroaryl ring system to form a further ring;
Z is selected from H, phenyl and pyridyl, where the phenyl and pyridyl groups are unsubstituted or substituted by one or more of the group selected from methyl, piperidinyl, benzyl, benzyl-4-OMe, benzyl-4-OCF₃, and benzyl-4-OSF₅;
L is selected from —CH₂NHCOCH₂CH₂— or —CH₂-phenyl-CH₂—, —CH₂—, —NH-pyrrolyl, imidazolyl, and thiazolyl.
or a pharmaceutically acceptable salt or solvate thereof.

2. The compound, or pharmaceutically acceptable salt or solvate thereof, according to Clause 1, wherein when X is a phenyl, pyridyl, quinazolinyl, or naphtyl group it is unsubstituted or is substituted by one of a first group of substituents and by one of a second group of substituents, where
the first group of substituents is H or Cl; and
the second group of substituents is NH₂, pyrrolyl, imidazolyl, tetrazolyl, CH₂NHCONH₂, and CH₂NHSO₂NH₂.

3. The compound, or pharmaceutically acceptable salt or solvate thereof, according to Clause 1 or Clause 2, wherein X is phenyl or pyridyl, optionally wherein X is pyridyl.

4. The compound, or pharmaceutically acceptable salt or solvate thereof, according to any one of the preceding clauses, wherein Y is benzimidazolyl or 5,6-dimethoxy benzimidazolyl.

5. The compound, or pharmaceutically acceptable salt or solvate thereof, according to any one of the preceding clauses, wherein L is —CH₂NHCOCH₂CH₂—.

6. The compound, according to any one of the preceding clauses, wherein the compound of formula I, or pharmaceutically acceptable salt or solvate thereof, is

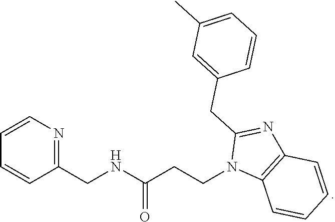 [SMILES: Cc1cccc(Cc2nc3ccccc3n2CCC(=O)NCc2ccccn2)c1]

7. Use of a compound of formula I as defined in any one of Clauses 1 to 6 or a salt or a solvate thereof, for the preparation of a medicament for the treatment of a bacterial infection caused by Mycobacterium abscessus, wherein the compound of formula I is administered sequentially, simultaneously or concomitantly with the other therapeutic agent.

8. A compound of formula I, as defined in any one of Clauses 1 to 6 or a salt or a solvate thereof, for use in the treatment of a bacterial infection caused by Mycobacterium abscessus.

9. A method of treatment of a bacterial infection caused by Mycobacterium abscessus, which method comprises the administration of a pharmaceutically effective amount of a compound of formula I as defined in any one of Clauses 1 to 6 or a salt or a solvate thereof, to a patient in need of such treatment.

10. Use of a compound of formula I as defined in any one of Clauses 1 to 6 or a salt or a solvate thereof, and another therapeutic agent, or a salt or solvate thereof, for the preparation of a medicament for the treatment of a bacterial infection caused by Mycobacterium abscessus, wherein the compound of formula I is administered sequentially, simultaneously or concomitantly with the other therapeutic agent.

11. A compound of formula I, as defined in any one of Clauses 1 to 6 or a salt or a solvate thereof, for use in the treatment of a bacterial infection caused by Mycobacterium abscessus, wherein the compound of formula I is administered sequentially, simultaneously or concomitantly with another therapeutic agent, or a salt or solvate thereof.

12. A method of treatment of a bacterial infection caused by Mycobacterium abscessus, which method comprises the administration of a pharmaceutically effective amount of a compound of formula I as defined in any one of Clauses 1 to 6 or a salt or a solvate thereof, and another therapeutic agent, or a salt or solvate thereof, to a patient in need of such treatment.

13. A method of identifying compounds that can bind to Mycobacterium abscessus F-ATP synthase subunit epsilon, comprising the steps of:
A) electronically screening stored spatial coordinates of a set of candidate compounds against the spatial coordinates comprising;
   i) C-terminal amino acid positions A107, R110, A111, R114 and A115 of the Mycobacterium abscessus F-ATP synthase subunit ε, which form a domain-domain interface, or binding pocket, with the N-terminal amino acid residues D46, D47, A48, A49, V50 and W61 of the M. abscessus F-ATP synthase subunit ε, and
   ii) amino acid positions γA42-A56 of subunit ε, which forms a protein-protein interface with M. abscessus F-ATP synthase subunit γ,
to identify compounds that can bind to said F-ATP synthase subunit ε, wherein the M. abscessus F-ATP synthase subunit E comprises the amino acid sequence set forth in SEQ ID NO: 1 and wherein the M. abscessus F-ATP synthase subunit γ comprises the amino acid sequence set forth in SEQ ID NO: 2; and
B) optionally determining whether identified compounds inhibit Mycobacterium abscessus F-ATP synthase subunit epsilon activity.

14. The method of claim 13, wherein a receptor pharmacophore model is developed on the Mycobacterium abscessus F-ATP synthase subunit epsilon residues in the interaction vicinity of amino acid positions A42-A56 of said Mycobacterium abscessus F-ATP synthase subunit γ.

15. The method of clause 13 or 14, further comprising molecular docking screening to further rank identified compounds.

16. The method of any one of clauses 13 to 15, wherein inhibition of Mycobacterium abscessus F-ATP synthase subunit epsilon activity will inhibit F-ATP synthase and M. abscessus growth.

### DRAWINGS

FIG. 1 depicts (A) the F₁F₀ ATP synthase is a molecular engine composed of the F₀ motor (a:c₉), the F₁-engine (α₃:β₃:γ:ε) and the peripheral stalk (b-δ:b′); and (B) amino acid sequence alignment of ε subunits from Homo sapiens, E. coli, M. abscessus sensu lato, M. abscessus subspecies bollettii, and M. abscessus subspecies massiliense were obtained from the UniProt database (The UniProt Consortium. 2012. Reorganizing the protein space at the Universal Protein Resource (UniProt). Nucleic Acids Res 40, D71-5) and imported into Jalview (Waterhouse, A. M. et al., Bioinformatics 2009, 25, 1189-1191). Alignment of the sequences was performed using ClustalWS (Thompson, J. D., Higgins, D. G. & Gibson, T. J., Nucleic Acids Res. 1994, 22, 4673-80). Thereafter, the calculation of the percentage of identity was performed and presented in darker to lighter shades of grey, representing the most homologous to the least homologous.

FIG. 2 depicts the nuclear magnetic resonance (NMR) spectrum of purified Mabε. (A) Size-exclusion chromatography results reveal a highly purified and homogenous sample eluted at 13.6 mL. Top 15% of the peak (shaded) was utilized for further downstream experiments. (Inset) 17% sodium dodecyl sulfate/polyacrylamide gel electrophoresis (SDS/PAGE) of purified recombinant Mabε (lane 2) with a protein marker in lane 1; and (B) 700-MHz ¹H-¹⁵N-heteronuclear single quantum coherence spectroscopy (HSQC) spectra of Mabε, which was recorded at 298 K on a Bruker Avance spectrometer. Backbone resonance assignments are indicated in a one-letter amino acid code and the sequence number.

FIG. 3 depicts the NMR solution structure of Mabε. (A) Superposition of the backbone traces from the final ensembles of 19 solution structures of Mabε, determined by NMR spectroscopy; and (B) A ribbon representation of the restrained energy minimized (REM) Mabε structure. The two α-helices represent the C-terminal domain (CTD).

FIG. 4 depicts the comparison of existing mycobacterial subunit ε structures. (A) Structural comparison of the NMR solution structure of Mabε (dark) and M. tuberculosis ε (Mtε) (light, PDB ID: 5Y10, Joon, S. et al., FEBS J. 2018, 285, 1111-1128); (B) Superimposition of the solution Mabε (dark) structure with the Msε crystal (light, PDB ID: 6FOC, Zhang, A. T. et al., Proc. Natl. Acad. Sci. U.S.A. 2019, 116, 4206-4211); and (C) The cryo-EM structure of Msε (light, PDB ID: 7JG6, Guo, H. et al., Nature 2021, 589, 143-147).

FIG. 5 depicts (A) surface representation of Mabε revealing the formation of a hydrophobic pocket inside the NTD and the molecular interaction network of the hydrophobic cleft (dark surface) and the CTD via the linker residues R87-D89. Amino acids involved in the molecular interaction network are represented in dark; (B) observed network of molecular interactions from residues on the hydrophobic pocket to CTD of Mtε via the NTD-CTD linker region. Residues involved in molecular interactions are highlighted as stick representation; and (C) an expanded view of the hydrophobic pocket, wherein residues involved in the molecular interaction network and important residues are highlighted.

FIG. 6 depicts (A) surface representation of Mtε showing the formation of a hydrophobic cleft inside the NTD of Mtε and molecular interaction network from hydrophobic cleft to CTD of Mtε via the linker region (S88-E89). The hydrophobic cleft is represented in dark colour and residues involving molecular interaction network are represented in sticks; (B) observed network of molecular interactions from residues on the hydrophobic cleft to CTD of Mtε via the NTD-CTD linker region. Residues involved in molecular interactions are highlighted as stick representation; and (C) an expanded view of the hydrophobic cleft wherein residues involved in the molecular interaction network and important residues are marked. These figures were taken from previously reported literature of NMR structure determination of Mtε (Shin, J. et al., FEBS J. 2018, 285, 1111-1128).

FIG. 7 depicts plots of the ¹⁵N relaxation data of Mabε. (A) R1 values; (B) R2 values; (C) ¹H-¹⁵N heteronuclear NOE values; (D) order parameter S2 values; and (E) R2/R1 ratios. NMR data were measured at 298 K on a 700-MHz NMR spectrometer. The average R2/R1 ratio is shown as dotted line, and the residues showing higher R2/R1 ratios than standard deviation range (shaded box) are labelled as one-letter code. The secondary structural elements are shown on the top of each panel.

FIG. 8 depicts the comparison of R2/R1 ratio of Mabε measured at a protein concentration of (A) 0.5 mM and (B) 0.3 mM. The average R2/R1 ratio is shown as dotted line and the residues showing higher R2/R1 ratios than the standard deviation range (shaded box) are labeled as one-letter code.

FIG. 9 depicts the native PAGE 4-20% gradient gel of the Mabε. The concentrations of MabE loaded on the gel were (1) 0.6 mM, (2) 1.5 mM and (3) 0.07 mM. All concentrations show a clear monomeric protein band slightly below 20 kDa. The first lane shows molecular marker proteins.

FIG. 10 depicts the NMR-titration experiment of Mabε and MgATP. Mabε titration with MgATP (molar ratio of 1:10). Dark peaks represent Mabε in the absence of MgATP and light peaks represent the protein in the presence of MgATP. No obvious changes in peak resonance were observed, indicating that Mabε does not bind MgATP.

FIG. 11 depicts that the Ramachandran plot shows that most of the residues are in favored and allowed regions. Only 10 residues that lacked template coordinates were seen in disallowed regions. As the loop regions are not critical for Mabγ-ε interactions, we have used this model for mapping the Mabγ-ε interactions.

FIG. 12 depicts the interaction interface of Mab's central stalk subunits γ-ε. Mainly, the γA42-A56 segment as well as residues L230-L234 mediate hydrophobic interactions with Mabε. In addition to these interactions, γR237 forms a polar contact with G67 main chain atoms of Mabε.

FIG. 13 depicts the Mabε pharmacophore model and binding pose of 3-(2-(3-methylbenzyl)-1H-benzo[d]imidazol-1-yl)-N-(pyridin-2-ylmethyl)propanamide (Ep1MabF1). (A) Receptor-based pharmacophore modelling MabE-γA42-A56 interaction interface. On the N-terminal side, two acceptors (A, labelled spheres) target residues E14 and S78. A hydrophobe (H, labelled spheres) feature anchors the Mabε residues V9 and V11. On the central side, 2 ring aromatic features (labelled as RA) anchor F69, L80 and V42 and a hydrophobic feature (H, labelled sphere) is positioned in the vicinity of methylene (—CH₂—) atoms of Mabε's S78 and K76. These six features along with the exclusion volumes (EV, white spheres) form the six featured receptor-based pharmacophore model of Mabε; (B) Chemical structure of Ep1MabF1 (IUPAC name is 3-(2-(3-methylbenzyl)-1H-benzo[d]imidazol-1-yl)-N-(pyridin-2-ylmethyl)propanamide); and (C) Ep1MabF1 with its three aromatic heterocyclic rings (3-methylbenzyl substituted benzimidazol-2-yl scaffold linked to pryid-2-yl ring by propionamide linker) mediates hydrophobic interactions with Mabε. The main scaffold benzimidazole was positioned towards Mabε amino acids F69 and V42, while its N1 atom is in close proximity (2.9 Å) with the hydroxyl group of S71. The 3-methylbenzyl ring on benzimidazole is anchored towards V77. The carbonyl “CO” group of propionamide is in H-bonding interaction with the hydroxyl atom of amino acid S78. The pyridyl group mediates the alkyl-aromatic interactions with residues V9, V11 and L80, while the “N” atom on pyridine was engaged in polar contacts with E14.

FIG. 14 depicts the growth and ATP synthesis inhibition by Ep1MabF1. (A) Intracellular inhibition of ATP synthesis of M. abscessus subsp. abscessus by the novel compound Ep1MabF1; (B) Effects of Ep1MabF1 of M. abscessus subsp. abscessus growth using cation-adjusted Mueller-Hinton (CAMH) medium; (C) Weighted Chemical Shift Perturbations (CSPs) for the ¹⁵N and ¹H resonance of Mabε after addition of Ep1MabF1. Residues showing CSPs above 0.015 ppm are labelled as one-letter code. The weighted CSPs between Mabε and Ep1MabF1 bound to Mabε for backbone ¹⁵N and ¹HN were calculated by the formula M8=[(ΔN/5)²+(ΔHN)²]^{0.5}; and (D, E) Surface (D) and ribbon (E) representations of the Ep1MabF1 interaction site based on the CSP results; the residues revealing more than 0.05 ppm are represented in black, while those showing CSP between 0.015 and 0.05 ppm are revealed in dark grey.

FIG. 15 depicts the NMR titration experiment of Mabε and Ep1MabF1. Mabε titration with Ep1MabF1 (molar ratio of 1:5). Dark peaks represent Mabε in the absence of Ep1MabF1 and light peaks represent the protein in the presence of Ep1MabF1 at a molar ratio of 1:5. Significantly changed peak resonances are displayed in dotted circles and labeled as one-letter code.

FIG. 16 depicts the differences of R2 values of Mabε in the presence and absence of Ep1MabF1. Residues showing values of R2 difference above average (dotted line) are labeled as one letter-code.

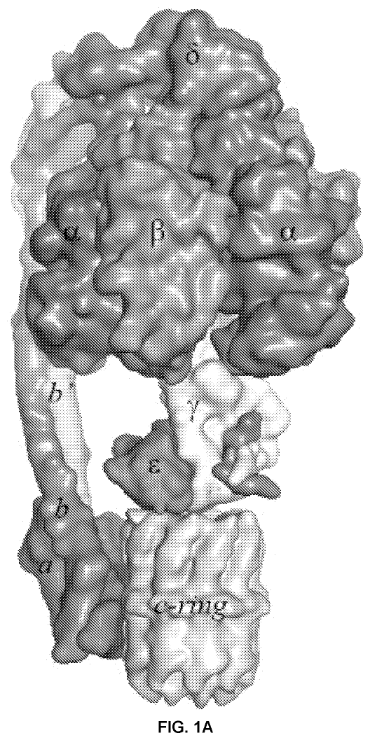

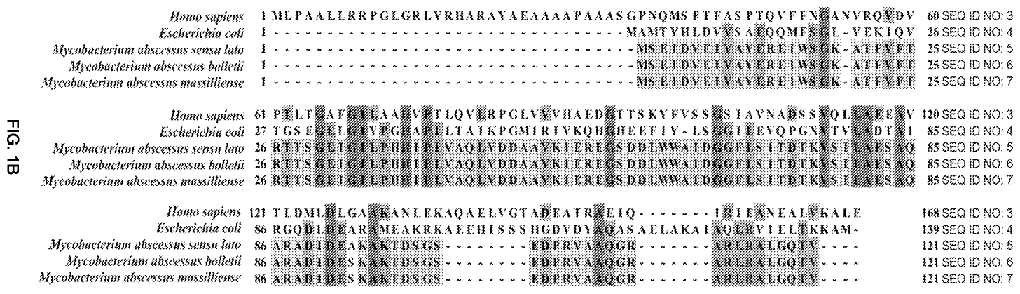

### DESCRIPTION

It has been surprisingly found that certain compounds are useful for the inhibition of the growth of Mycobacterium abscessus' F-ATP synthase. Thus in a first aspect of the invention, there is provided a compound of formula I:

X-L-Y—Z I
where:
X is selected from phenyl, pyridyl, quinazolinyl, or naphtyl, where the phenyl, pyridyl, quinazolinyl, or naphtyl group is unsubstituted or substituted by one or more of the group selected from Cl, NH₂, pyrrolyl, imidazolyl, tetrazolyl, CH₂NHCONH₂, and CH₂NHSO₂NH₂;
Y is a heteroaryl ring system selected from benzimidazolyl, benzothiazolyl, benzofuranyl, quinazolinyl, and naphthyl, which groups are unsubstituted or substituted by one or more substituents selected from the group consisting of OMe, —O—CH₂—O—, and —O(CH₂)₂O—, where the oxygen atoms in the latter two groups are attached to different atoms on the heteroaryl ring system to form a further ring;
Z is selected from H, phenyl and pyridyl, where the phenyl and pyridyl groups are unsubstituted or substituted by one or more of the group selected from methyl, piperidinyl, benzyl, benzyl-4-OMe, benzyl-4-OCF₃, and benzyl-4-OSF₅;
L is selected from —CH₂NHCOCH₂CH₂— or —CH₂-phenyl-CH₂—, —CH₂—, —NH-pyrrolyl, imidazolyl, and thiazolyl.
or a pharmaceutically acceptable salt or solvate thereof.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

As used herein, the singular forms “a,” “an,” and “the” include plural referents unless the context clearly dictates otherwise. Thus, for example, reference to “a composition” includes mixtures of two or more such compositions, reference to “the catalyst” includes mixtures of two or more such catalysts, and the like.

References herein (in any aspect or embodiment of the invention) to compounds of formula I includes references to such compounds per se, to tautomers of such compounds, as well as to pharmaceutically acceptable salts or solvates, or pharmaceutically functional derivatives of such compounds.

Pharmaceutically acceptable salts that may be mentioned include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of formula I in the form of a salt with another counter-ion, for example using a suitable ion exchange resin.

Examples of pharmaceutically acceptable salts include acid addition salts derived from mineral acids and organic acids, and salts derived from metals such as sodium, magnesium, or preferably, potassium and calcium.

Examples of acid addition salts include acid addition salts formed with acetic, 2,2-dichloroacetic, adipic, alginic, aryl sulphonic acids (e.g. benzenesulphonic, naphthalene-2-sulphonic, naphthalene-1,5-disulphonic and p-toluenesulphonic), ascorbic (e.g. L-ascorbic), L-aspartic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, gluconic (e.g. D-gluconic), glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic (e.g. (−)-L-malic), malonic, (±)-DL-mandelic, metaphosphoric, methanesulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, tartaric (e.g. (+)-L-tartaric), thiocyanic, undecylenic and valeric acids.

Particular examples of salts are salts derived from mineral acids such as hydrochloric, hydrobromic, phosphoric, metaphosphoric, nitric and sulphuric acids; from organic acids, such as tartaric, acetic, citric, malic, lactic, fumaric, benzoic, glycolic, gluconic, succinic, arylsulphonic acids; and from metals such as sodium, magnesium, or preferably, potassium and calcium.

As mentioned above, also encompassed by formula I are any solvates of the compounds and their salts. Preferred solvates are solvates formed by the incorporation into the solid state structure (e.g. crystal structure) of the compounds of the invention of molecules of a non-toxic pharmaceutically acceptable solvent (referred to below as the solvating solvent). Examples of such solvents include water, alcohols (such as ethanol, isopropanol and butanol) and dimethylsulphoxide. Solvates can be prepared by recrystallising the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGE), differential scanning calorimetry (DSC) and X-ray crystallography.

The solvates can be stoichiometric or non-stoichiometric solvates. Particularly preferred solvates are hydrates, and examples of hydrates include hemihydrates, monohydrates and dihydrates.

For a more detailed discussion of solvates and the methods used to make and characterise them, see Bryn et al., Solid-State Chemistry of Drugs, Second Edition, published by SSCI, Inc of West Lafayette, IN, USA, 1999, ISBN 0-967-06710-3.

Compounds of formula I, as well as pharmaceutically acceptable salts, solvates and pharmaceutically functional derivatives of such compounds are, for the sake of brevity, hereinafter referred to together as the “compounds of formula I”.

Compounds of formula I may contain double bonds and may thus exist as E (entgegen) and Z (zusammen) geometric isomers about each individual double bond. All such isomers and mixtures thereof are included within the scope of the invention.

Compounds of formula I may exist as regioisomers and may also exhibit tautomerism. All tautomeric forms and mixtures thereof are included within the scope of the invention.

Compounds of formula I may contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention. Further embodiments of the invention that may be mentioned include those in which the compound of formula I is isotopically labelled. However, other, particular embodiments of the invention that may be mentioned include those in which the compound of formula I is not isotopically labelled.

The term “isotopically labelled”, when used herein includes references to compounds of formula I in which there is a non-natural isotope (or a non-natural distribution of isotopes) at one or more positions in the compound. References herein to “one or more positions in the compound” will be understood by those skilled in the art to refer to one or more of the atoms of the compound of formula I. Thus, the term “isotopically labelled” includes references to compounds of formula I that are isotopically enriched at one or more positions in the compound.

The isotopic labelling or enrichment of the compound of formula I may be with a radioactive or non-radioactive isotope of any of hydrogen, carbon, nitrogen, oxygen, sulfur, fluorine, chlorine, bromine and/or iodine. Particular isotopes that may be mentioned in this respect include ²H, ³H, ¹¹C, ¹³C, ¹⁴C ¹³N, ¹⁵N, ¹⁵O ¹⁷O, ¹⁸O, ³⁵S, ¹⁸F, ³⁷Cl, ⁷⁷Br, ⁸²Br and ¹²⁵I). When the compound of formula I is labelled or enriched with a radioactive or nonradioactive isotope, compounds of formula I that may be mentioned include those in which at least one atom in the compound displays an isotopic distribution in which a radioactive or non-radioactive isotope of the atom in question is present in levels at least 10% (e.g. from 10% to 5000%, particularly from 50% to 1000% and more particularly from 100% to 500%) above the natural level of that radioactive or non-radioactive isotope.

In embodiments of the invention that may be mentioned herein, the compound, or pharmaceutically acceptable salt or solvate thereof of formula I may be one in which, when X is a phenyl, pyridyl, quinazolinyl, or naphtyl group it is unsubstituted or is substituted by one of a first group of substituents and by one of a second group of substituents, where
the first group of substituents is H or Cl; and
the second group of substituents is NH₂, pyrrolyl, imidazolyl, tetrazolyl, CH₂NHCONH₂, and CH₂NHSO₂NH₂.

In further embodiments of the invention that may be mentioned herein, the compound of formula I, or pharmaceutically acceptable salt or solvate thereof may be one where X is phenyl or pyridyl, optionally wherein X is pyridyl.

In further embodiments of the invention that may be mentioned herein, the compound of formula I, or pharmaceutically acceptable salt or solvate thereof may be one where Y is benzimidazolyl or 5,6-dimethoxy benzimidazolyl.

In further embodiments of the invention that may be mentioned herein, the compound of formula I, or pharmaceutically acceptable salt or solvate thereof may be one where L is —CH₂NHCOCH₂CH₂—.

In particular embodiments of the invention that may be mentioned herein, the compound of formula I, or pharmaceutically acceptable salt or solvate thereof, may be:

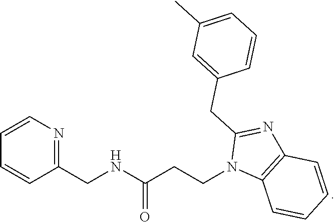 [SMILES: Cc1cccc(Cc2nc3ccccc3n2CCC(=O)NCc2ccccn2)c1]

The compound for use mentioned in the above-mentioned aspect of the invention may be utilised in a method of medical treatment. Thus, according to further aspects of the invention, there is provided the following further aspects of invention.

(AA) Use of a compound of formula I as described herein or a salt or a solvate thereof, for the preparation of a medicament for the treatment of a bacterial infection caused by Mycobacterium abscessus, wherein the compound of formula I is administered sequentially, simultaneously or concomitantly with the other therapeutic agent.

(AB) A compound of formula I, as described herein or a salt or a solvate thereof, for use in the treatment of a bacterial infection caused by Mycobacterium abscessus.

(AC) A method of treatment of a bacterial infection caused by Mycobacterium abscessus, which method comprises the administration of a pharmaceutically effective amount of a compound of formula I as described herein or a salt or a solvate thereof, to a patient in need of such treatment.

(AD) Use of a compound of formula I as described herein or a salt or a solvate thereof, and another therapeutic agent, or a salt or solvate thereof, for the preparation of a medicament for the treatment of a bacterial infection caused by Mycobacterium abscessus, wherein the compound of formula I is administered sequentially, simultaneously or concomitantly with the other therapeutic agent.

(AE) A compound of formula I, as described herein or a salt or a solvate thereof, for use in the treatment of a bacterial infection caused by Mycobacterium abscessus, wherein the compound of formula I is administered sequentially, simultaneously or concomitantly with another therapeutic agent, or a salt or solvate thereof.

(AF) A method of treatment of a bacterial infection caused by Mycobacterium abscessus, which method comprises the administration of a pharmaceutically effective amount of a compound of formula I as described herein or a salt or a solvate thereof, and another therapeutic agent, or a salt or solvate thereof, to a patient in need of such treatment.

For the avoidance of doubt, in the context of the present invention, the term “treatment” includes references to therapeutic or palliative treatment of patients in need of such treatment, as well as to the prophylactic treatment and/or diagnosis of patients which are susceptible to the relevant disease states.

The terms “patient” and “patients” include references to mammalian (e.g. human) patients. As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment or a subject with a disease or disorder. However, in other embodiments, the subject can be a normal subject. The term does not denote a particular age or sex. Thus, adult and newborn subjects, whether male or female, are intended to be covered.

The term “effective amount” refers to an amount of a compound, which confers a therapeutic effect on the treated patient (e.g. sufficient to treat or prevent the disease). The effect may be objective (i.e. measurable by some test or marker) or subjective (i.e. the subject gives an indication of or feels an effect).

Compounds of formula I may be administered by any suitable route, but may particularly be administered orally, intravenously, intramuscularly, cutaneously, subcutaneously, transmucosally (e.g. sublingually or buccally), rectally, transdermally, nasally, pulmonarily (e.g. tracheally or bronchially), topically, by any other parenteral route, in the form of a pharmaceutical preparation comprising the compound in a pharmaceutically acceptable dosage form. Particular modes of administration that may be mentioned include oral, intravenous, cutaneous, subcutaneous, nasal, intramuscular or intraperitoneal administration.

Compounds of formula I will generally be administered as a pharmaceutical formulation in admixture with a pharmaceutically acceptable adjuvant, diluent or carrier, which may be selected with due regard to the intended route of administration and standard pharmaceutical practice. Such pharmaceutically acceptable carriers may be chemically inert to the active compounds and may have no detrimental side effects or toxicity under the conditions of use. Suitable pharmaceutical formulations may be found in, for example, Remington The Science and Practice of Pharmacy, 19th ed., Mack Printing Company, Easton, Pennsylvania (1995). For parenteral administration, a parenterally acceptable aqueous solution may be employed, which is pyrogen free and has requisite pH, isotonicity, and stability. Suitable solutions will be well known to the skilled person, with numerous methods being described in the literature. A brief review of methods of drug delivery may also be found in e.g. Langer, Science (1990) 249, 1527.

Otherwise, the preparation of suitable formulations may be achieved routinely by the skilled person using routine techniques and/or in accordance with standard and/or accepted pharmaceutical practice.

The amount of compound of formula I in any pharmaceutical formulation used in accordance with the present invention will depend on various factors, such as the severity of the condition to be treated, the particular patient to be treated, as well as the compound(s) which is/are employed. In any event, the amount of compound of formula I in the formulation may be determined routinely by the skilled person.

For example, a solid oral composition such as a tablet or capsule may contain from 1 to 99% (w/w) active ingredient; from 0 to 99% (w/w) diluent or filler; from 0 to 20% (w/w) of a disintegrant; from 0 to 5% (w/w) of a lubricant; from 0 to 5% (w/w) of a flow aid; from 0 to 50% (w/w) of a granulating agent or binder; from 0 to 5% (w/w) of an antioxidant; and from 0 to 5% (w/w) of a pigment. A controlled release tablet may in addition contain from 0 to 90% (w/w) of a release-controlling polymer.

A parenteral formulation (such as a solution or suspension for injection or a solution for infusion) may contain from 1 to 50% (w/w) active ingredient; and from 50% (w/w) to 99% (w/w) of a liquid or semisolid carrier or vehicle (e.g. a solvent such as water); and 0-20% (w/w) of one or more other excipients such as buffering agents, antioxidants, suspension stabilisers, tonicity adjusting agents and preservatives.

Depending on the disorder, and the patient, to be treated, as well as the route of administration, compounds of formula I may be administered at varying therapeutically effective doses to a patient in need thereof.

However, the dose administered to a mammal, particularly a human, in the context of the present invention should be sufficient to effect a therapeutic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the potency of the specific compound, the age, condition, body weight, sex and response of the patient to be treated, and the stage/severity of the disease.

Administration may be continuous or intermittent (e.g. by bolus injection). The dosage may also be determined by the timing and frequency of administration. In the case of oral or parenteral administration the dosage can vary from about 0.01 mg to about 1000 mg per day of a compound of formula I.

In any event, the medical practitioner, or other skilled person, will be able to determine routinely the actual dosage, which will be most suitable for an individual patient. The above-mentioned dosages are exemplary of the average case; there can, of course, be individual instances where higher or lower dosage ranges are merited, and such are within the scope of this invention.

The aspects of the invention described herein (e.g. the above-mentioned compounds, combinations, methods and uses) may have the advantage that, in the treatment of the conditions described herein, they may be more convenient for the physician and/or patient than, be more efficacious than, be less toxic than, have better selectivity over, have a broader range of activity than, be more potent than, produce fewer side effects than, or may have other useful pharmacological properties over, similar compounds, combinations, methods (treatments) or uses known in the prior art for use in the treatment of those conditions or otherwise.

The identification of compounds useful for methods of medical treatment of Mycobacterium abscessus infection may be realised by the use of certain methods. Thus, according to further aspects of the invention, there is provided the following:

(BA) A method of identifying compounds that can bind to Mycobacterium abscessus F-ATP synthase subunit epsilon, comprising the steps of:
A) electronically screening stored spatial coordinates of a set of candidate compounds against the spatial coordinates comprising;
i) C-terminal amino acid positions A107, R110, A111, R114 and A115 of the Mycobacterium abscessus F-ATP synthase subunit ε, which form a domain-domain interface, or binding pocket, with the N-terminal amino acid residues D46, D47, A48, A49, V50 and W61 of the M. abscessus F-ATP synthase subunit ε, and
ii) amino acid positions γA42-A56 of subunit ε, which forms a protein-protein interface with M. abscessus F-ATP synthase subunit γ,
to identify compounds that can bind to said F-ATP synthase subunit ε, wherein the M. abscessus F-ATP synthase subunit E comprises the amino acid sequence set forth in SEQ ID NO: 1 and wherein the M. abscessus F-ATP synthase subunit γ comprises the amino acid sequence set forth in SEQ ID NO: 2; and
B) optionally determining whether identified compounds inhibit Mycobacterium abscessus F-ATP synthase subunit epsilon activity.

(BB) The method described in (BA), wherein a receptor pharmacophore model is developed on the Mycobacterium abscessus F-ATP synthase subunit epsilon residues in the interaction vicinity of amino acid positions A42-A56 of said Mycobacterium abscessus F-ATP synthase subunit γ.

(BC) The method described in (BA) or (BB), further comprising molecular docking screening to further rank identified compounds.

(BD) The method described in any of (BA) to (BC), wherein inhibition of Mycobacterium abscessus F-ATP synthase subunit epsilon activity will inhibit F-ATP synthase and M. abscessus growth.

Further aspects and embodiments of the invention will now be discussed by reference to the following non-limiting examples.

### EXAMPLES

Materials Kapa HiFi DNA polymerase was purchased from KAPA Biosystems (Wilmington, MA, USA), and Ni²⁺-NTA chromatography resin was obtained from Qiagen (Hilden, Germany). Enzymatic digestion was performed using restriction enzymes from New England BioLabs. Chemicals from Bio-Rad (Hercules, CA, USA) were used for SDS/PAGE. All other chemicals of analytical grade were obtained from BIOMOL (Hamburg, Germany), Merck (Darmstadt, Germany), Sigma or Serva (Heidelberg, Germany). BacTiter-Glo microbial cell viability assay was purchased from Promega. Amicon® Ultra-4 Centrifugal Filters (10 kDa molecular mass cut-off, spin concentrators) were purchased from MilliporeSigma, Burlington, MA, USA. Resource™ Q column, 6 mL was purchased from GE Healthcare, Chicago, IL, USA. Resource™ Q column, 1 mL was purchased from GE Healthcare, Sweden. HiLoad 16/600 Superdex 75 prep-grade column was purchased from GE Healthcare. Cation-adjusted Mueller-Hinton (CAMH) broth was purchased from BD Difco.

### Analytical Techniques

### NMR Spectroscopy

All NMR experiments were carried out on a BrukerAvance 700-MHz spectrometer, which was equipped with a cryoprobe at 298 K.

### Example 1. Cloning, Expression and Purification of Mabε

The mycobacterial F-ATP synthase subunit ε is essential for the growth and viability of the bacterium. Besides its central role in the formation of the currency of life, ATP, understanding of the specific epitopes of mycobacterial subunit ε in the regulation of latency of ATP hydrolysis, and preventing wastage of ATP during metabolic stress phases, pave the way for new M. tuberculosis F-ATP synthase inhibitors binding to ε.

The gene atpC, which contains the coding sequence of MabE(S2-V121), was amplified using the Mab atp-operon as a template. Amplification of the atpC gene was performed with the following primers: 5′-TAA GAA GGA GAT ATA CCA TGT CCG AGA TTG ATG TCG AGA TCG TCG-3′ and 5′-CGG AGC TCG AAT TCG GAT CCC TAA ACC GTC TGG CCG AG-3′. The linearized pYUB1049 vector (Bashiri, G. et al., PLoS one 2010, 5, e15803) was amplified, and the two DNA fragments were incorporated utilizing the NEBuilder® HiFi DNA Assembly Cloning as per the manufacturer's protocol. DNA sequencing (BioBasic, Asia Pacific Pte Ltd, Singapore) was performed to verify the plasmid. After obtaining the plasmid carrying the gene of interest, site-directed mutagenesis was performed to incorporate a N-terminal His₆-tag with the following primers: 5′-ACC ATG CAT CAC CAT CAC CAT CAT TCC GAG ATT GAT GTC GAG ATC G-3′ and 5′-CGG AAT GAT GGT GAT GGT GAT GCA TGG TAT ATC TCC TTC TTA AAG TTA AAC-3′. Unmethylated DNA was subsequently removed through DpnI treatment. Finally, the plasmid was transformed into Escherichia coli TOP10 cells. Plasmid sequencing was once again performed to ensure the incorporation of the His₆-tag at the N terminus of the gene.

To produce the recombinant His₆-tagged Mabε, Escherichia coli C41 cells were incubated in hygromycin-containing (150 μg·mL⁻¹) Luria-Bertani (LB) medium at 37° C. with shaking of 180 rpm. After an optical density OD₆₀₀ of 0.6-0.7 was achieved, Mabε production was obtained by adding of isopropyl (thio)-β-D-galactopyranoside (IPTG) to a final concentration of 1 mM. After overnight incubation at 18° C., the cells were harvested at 8500 g for 13 min, 4° C. Subsequently, cells were lysed on ice by sonication (Bandelin, KE76 tip) for 3×1 min in buffer A (50 mM Tris/HCl, pH 7.5, 200 mM NaCl, 2 mM Pefabloc^{SC} (4-(2-aminoethyl)benzenesulfonyl fluoride hydrochloride) (BIOMOL) and 10% glycerol (v/v)). Precipitated material was separated by centrifugation (Eppendorf 581OR Centrifuge, Eppendorf, Hamburg, Germany) at 15,000 g for 30 min at 4° C., and the supernatant was filtered (0.45 μm; Millipore). The latter was further incubated with Ni²⁺-NTA resin (Qiagen) column for 1 h at 4° C. to isolate Mabε. The His₆-tagged proteins were eluted with an imidazole gradient (0-400 mM) in buffer A. Fractions containing Mabε diluted in buffer A to 50 mM NaCl and subsequently applied on a Resource™ Q column, 1 mL. The purity was improved by a salt gradient using the following buffers: 50 mM Tris/HCl and pH 7.5, and 50 mM Tris/HCl, pH 7.5 and 1 M NaCl. Mabε eluted in the flow-through, whereas contaminants were bound to the column. Eluted Mabε was concentrated using Amicon® Ultra-4 Centrifugal Filters (10 kDa molecular mass cut-off, spin concentrators) before applying on a Superdex 75 HR 10/30 column (GE Healthcare), which was equilibrated with a buffer containing 50 mM Tris/HCl, pH 7.5 and 150 mM NaCl. For large-scaled purification, a Resource™ Q column, 6 mL and HiLoad 16/600 Superdex 75 prep-grade column were respectively used instead.

To produce ¹⁵N and ¹³C-¹⁵N labelled Mabε for NMR spectroscopy experiments, freshly transformed E. coli C41 (DE3) cells were plated on LB agar from which a single colony was selected to prepare a 50 ml LB seed culture supplemented with 150 μg/ml hygromycin B. Cells were incubated overnight at 37° C. with an orbital shaking of 180 rpm. Subsequently, the culture was centrifuged at 4,000 g for 10 mins at 4° C. to pellet the cells. The pelleted cells were washed and re-suspended in 2 L M9 minimal media supplemented with 0.1 mM CaCl₂, 2 mM MgSO₄, 10 g/L D-glucose, 1 mM trace elements (Cu²⁺, Zn²⁺, Mn²⁺, Co²⁺, Ni²⁺, MoO⁴⁻), 30 μM FeCl₃, 5 ng/L Thiamine HCl and hygromycin B at a starting optical density of 600 nm (OD₆₀₀) of 0.1. The cultures were left to incubate at 37° C. with an orbital shaking of 180 rpm until an OD₆₀₀ of 0.6-0.7 was achieved. Next, ¹⁵NH₄Cl or a combination of ¹⁵NH₄Cl with ¹³C₆-D-glucose was used for the preparation of uniformly ¹⁵N and ¹³C-¹⁵N-isotopically labelled protein, respectively. The cells were induced with Isopropyl β-D-1-thiogalactopyranoside (IPTG) to a final concentration of 1 mM and left to incubate overnight at 18° C. and shaking of 180 rpm prior to harvesting. All labelled materials were purchased from Cambridge Isotope Laboratories. For ¹³C-nuclear overhauser effect spectroscopy (NOESY) experiments, buffers used were prepared in 100% D₂O.

The amino acid sequences of Mabε and Mab γ are shown in Table 1.

TABLE 1
Amino acid sequences of Mabε and Mabγ.

| Protein | Amino acid sequence |
|---|---|
| Mabε SEQ | MSEIDVEIVAVEREIWSGKATFVFTRTTSGEIGILPHHI |
| ID NO: 1 | PLVAQLVDDAAVKIEREGSDDLWWAIDGGFLSITDTKVS |
| | ILAESAQARADIDEAKAKTDSGSEDPRVAAQGRARLRAL |
| | GQTV |
| Mabγ SEQ | MGAQIRELRRRIKSAGAIKKITKAQELIATSRIAKAQAR |
| ID NO: 2 | VVAARPYATEITNVLTALADDAALDHPLLVERPEPKRAG |
| | VLIVSSDRGLCGGYNANVLRVAEELYALLREQGKTPVVY |
| | VVGRKALNYYSFRNRKVTEAWTGFSERPEYASAQKIADT |
| | LVEAFLAGADDEGDDPGLDGILGVDELHIVYTEFKSMLT |
| | QAAVAKRIAPMEVEYVGEAAGPTTQYSFEPDATTLFGAL |
| | LPRYLATRVYAALLEAAASESASRRRAMKAATDNADELI |
| | KGLTLEANGARQAQITQEISEIVGGVNALADAAGH |

### Example 2. Atomic Solution Structure of the M. abscessus F-ATP Synthase Subunit ε

### NMR Spectroscopy Data Collection and Analysis

0.5 mM of uniformly labelled ¹⁵N and ¹³C/¹⁵N Mabε, prepared in buffer containing 50 mM Tris/HCl, pH 7.5, 150 mM NaCl, 0.01% NaN₃ and 10% D₂O, was used in solution NMR experiments. Conventional 2D and 3D heteronuclear NMR data and 3D triple resonance spectra of this sample were recorded. The latter were collected by the nonuniform sampling (NUS, Rovnyak, D. et al., J. Magn. Reson. 2004, 170, 15-21) mode of the indirect dimension as 20-25% sampling rates and reconstructed using SMILE plug-in (Ying, J. et al., J. Biomol. NMR 2017, 68, 101-118) of NMRPipe/NMRDraw software (Delaglio, F. et al., J. Biomol. NMR 1995, 6, 277-293). The 2D ¹H-¹⁵N HSQC experiment was done in a conventional uniform sampling manner. All NMR experiments were carried out on a Bruker Avance 700-MHz spectrometer, which was equipped with a cryoprobe at 298 K. The data were processed/analysed using NMRPipe/NMRDraw (Delaglio, F. et al., J. Biomol. NMR 1995, 6, 277-293) and analysed by SPARKY (Goddard, T. & Kneller, D., Sparky 3. San Francisco, CA: University of California; 2008), respectively. Assignments of the protein backbone ¹⁵N, ¹HN, ¹³Ca, ¹³Cp and ¹³C′ chemical shifts of Mabε were carried out based on HNCACB, CBCA(CO)NH, HNCA, HN(CO)CA, HNCO and ¹H-¹⁵N—HSQC spectra. The chemical shifts of side-chain and missing backbone resonances were obtained from a combination of ¹H-¹³C—HCCH-total correlation spectroscopy (TOCSY) and ¹³C/¹⁵N-simultaneous nuclear overhauser effect spectroscopy (NOESY)-HSQC spectra. Distance restraints for structure determination were derived from analysis of ¹³C/¹⁵N-simultaneous NOESY-HSQC and ¹H-¹³C-NOESY-HSQC spectra recorded in 100-ms mixing time.

### Structure Calculation and Refinement of Mabε

Solution structures of Mabε were calculated by simulated annealing in torsion angle space with a combination of the programs CYANA 2.1 (Guntert, P., Eur. Biophys. J. 2009, 38, 129-143) and CNS 1.2 (Brunger, A. T. et al., Acta Cryst. 1998, 54, 905-921; and Brunger, A. T., Nat. Protoc. 2007, 2, 2728-2733). Nuclear Overhauser effect (NOE) distance constraints were extracted from ¹³C/¹⁵N-simultaneous NOESY-HSQC (100-ms mixing time) and ¹³C-edited NOESY-HSQC (120-ms mixing time) spectra of uniformly ¹³C/¹⁵N-labeled samples of Mabε in 90% H₂O/10% D₂O and 100% D₂O conditions, respectively. The secondary structure was predicted by TALOS+ program (Shen, Y. et al., J. Biomol. NMR 2009, 44, 213-223) based on the results of the analysis of chemical shifts of the main-chain N, HA, CA and C atoms and sequential (¦i_i ¦=1) and short-range (¦i_i ¦<5) NH—NH and NH-aliphatic contacts on a ¹H/¹⁵N-NOESY-HSQC lanes of ¹³C/¹⁵N-simultaneous NOESY-HSQC spectra. Dihedral angle (phi, psi) restraints were also calculated from chemical shifts using TALOS+, and hydrogen bond restraints were obtained based on the protein structure during structure calculations. NOE cross-peaks on NOESY spectra were classified based on their intensities and were applied with an upper distance limit of 2.8 Å (strong), 4.0 Å (medium), 5.0 Å (weak) and 5.5 Å (very weak). An additional 0.5 Å was added for NOEs that involved methylene and methyl groups. A total of 1,000 conformers were generated as initial structures by CYANA 2.1 from 2088 NOE and 185 backbone dihedral angle constraints. After calculation of initial structure, lowest 200 conformers were selected by their target function for further refinement using CNS 1.2. 136 backbone hydrogen bonds were identified on the basis of initial structures and included in the final stage of the calculation. The final structure was refined using a simulated annealing protocol with a combination of torsion angle space and Cartesian coordinate dynamics (Nilges, M., Clore, G. M. & Gronenborn, A. M., FEBS Lett. 1988, 239, 129-136) as described previously. Finally, 19 structures were selected by their total energy values for display and structural analysis. MOLMOL (Koradi, R., Billeter, M. & Wuthrich, K., J. Mol. Graph. 1996, 14, 51-55) and PYMOL programs (Schrödinger, LLC. The PyMOL molecular graphics system, Version 2.0. New York, NY: Schrödinger, LLC; 2017) were used for structure visualization and PROCHECK-NMR (Laskowski, R. A. et al., J. Biomol. NMR 1996, 8, 477-486). The 19 NMR ensembles, restrained energy minimized (REM) structure and assigned chemical shift data have been deposited in the Protein Data Bank with the PDB ID: 7XKZ and BioMagnetic Resonance Data Bank with the BMRB ID: 36485, respectively.

### Results and Discussion

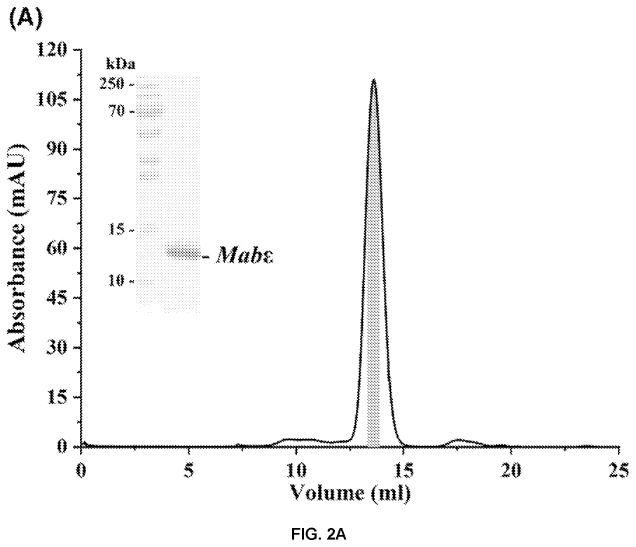

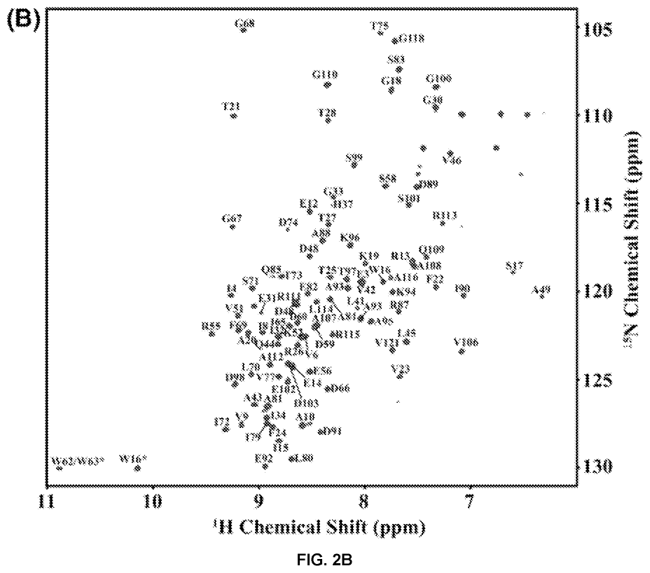

The recombinant unlabelled, ¹⁵N- and ¹³C¹⁵N-labeled Mabε(S2-V121) (120 amino acids) were generated as described in Example 1. The recombinant protein was purified using a two-step purification, including affinity chromatography and size-exclusion chromatography, where Mabε eluted at 13.6 mL on a Superdex™ 75, which corresponds to a monomeric form (FIG. 2A). The fractions forming 15% of the peak containing labelled Mabε were pooled and identified using a 17% SDS/PAGE (FIG. 2A).

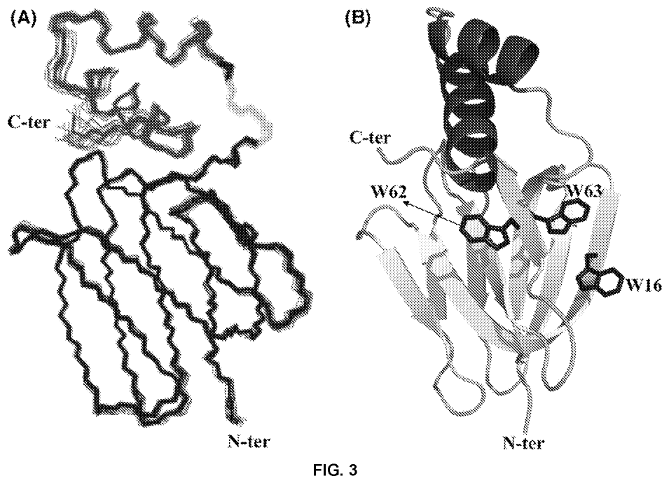

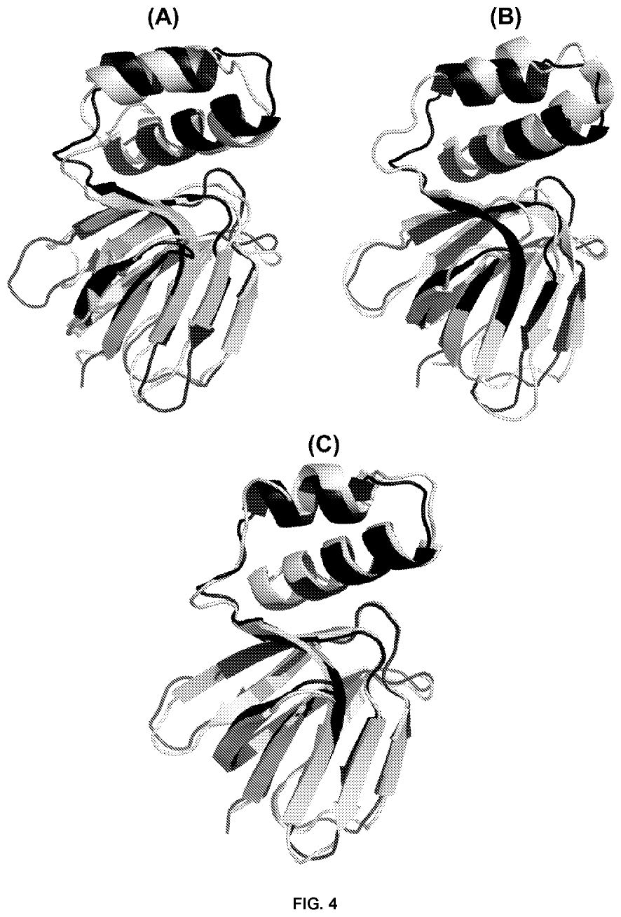

A high quality of 2D ¹H-¹⁵N HSQC spectrum of recombinant Mabε was obtained, revealing proper folding and monodispersity of the protein (FIG. 2B). Among the 117 nonproline residues of Mabε, ¹H and ¹⁵N chemical shifts of 101 residues were assigned except for 16 residues, which did not appear in the ¹H-¹⁵N HSQC, 3D HNCA or HNCACB spectra. However, combining with CBCA(CO)NH, HN(CO) CA, HCCH-TOCSY, and ¹³C/¹⁵N-simultaneous NOESY-HSQC spectra, 116 backbone Cα resonances could be assigned as 96.7% completeness for 120 Cα resonances. Finally, 95.2% of resonances were assigned including backbone and side chains based on the NMR experimental data. The assignments of the resolved backbone residues are shown in FIG. 2B. The solution structure of Mabε was determined based on a total of 2088 NOEs, 136 hydrogen bonds and 185 dihedral angle restraints (FIG. 3). The ensemble of 19 low-energy structures and restrained energy minimized (REM) average structure is shown in FIGS. 3A-B. The root-mean-square deviation (RMSD) values for E3-T120, relative to the REM coordinate of 19 conformers, were 0.39 Å for the backbone atoms and 0.97 Å for all heavy atoms (Table 2), revealing a highly converged structure in solution. The structural statistics for the NMR ensemble are given in Table 2. There are no distance violations >0.5 Å or dihedral angle violations >5°. Restrained energy minimization calculations using PROCHECK-NMR showed that 84.9%, 14.2% and 0.9% of the residues lie in the most favoured, additionally allowed and generally allowed regions, respectively. The overall structure revealed a well-conserved β-barrel for the NTD and a helix-loop-helix fold for the CTD. Both domains are connected by a short linker segment formed by the amino acids R87-D91, similar to the overall domain arrangement determined for M. tuberculosis ε (Mtε; FIG. 4A, Joon, S. et al., FEBS J. 2018, 285, 1111-1128) and Msε (Guo, H. et al., Nature 2021, 589, 143-147), respectively (FIG. 4). The overall RMSD values of MabE with Mtε (PDB ID: 5YIO, Wong, C. F. et al., FEBS J. 2020, 288, 818-836) and Msε (PDB ID: 7JG6, Guo, H. et al., Nature 2021, 589, 143-147) are about 1.78 Å, 2.63 Å (Msε crystal structure, PDB ID: 6FOC, Zhang, A. T. et al., Proc. Natl. Acad. Sci. U.S.A. 2019, 116, 4206-4211) and 0.91 Å (Msε cryo-EM structure, PDB ID: 7JG6, Guo, H. et al., Nature 2021, 589, 143-147) for 117 backbone Cα, respectively. Although the overall backbone fold and secondary structure of Mabε are similar to the previously reported Mtε and Msε ones, structural differences were observed in the loop region and especially in the NTD. Interestingly, structural differences between Mabε and the Msε crystal structure (Zhang, A. T. et al., Proc. Natl. Acad. Sci. U.S.A. 2019, 116, 4206-4211) were observed, especially regarding the shorter second helix in the CTD and the lack of one β-strand (β-4) (FIG. 4B), which in part may be due to the moderate resolution of the 4.0 Å Msε crystal structure (Zhang, A. T. et al., Proc. Natl. Acad. Sci. U.S.A. 2019, 116, 4206-4211). No significant differences were observed between the solution NMR Mabε structure and the Msε cryo-EM structure (Guo, H. et al., Nature 2021, 589, 143-147, FIG. 4C).

TABLE 2
Structural statistics of the atomic
solution NMR structures of Mabε.
Number of NOE constraints

| | |
|---|---|
| All | 2088 |
| Intraresidues \|i_j\| = 0 | 624 |
| Sequential, \|i_j\| = 1 | 442 |
| Medium range, 1 < \|i_j\| < 5 | 244 |
| Long range, \|i_j\| >= 5 | 778 |
| Number of hydrogen bond constraints | 136 |
| Number of dihedral angle constraints | 185 |
| Number of constraint violations (>0.5 Å) | 0 |
| Number of angle violations (>5°) | 0 |

Energies (kcal · mol⁻¹)

| | |
|---|---|
| Eₜₒₜₐₗ | 46.05 ± 0.66 |
| E_{NOE} | 1.05 ± 0.21 |
| E_{cdih} | 0.07 ± 0.02 |
| E_{bond} + E_{angle} + Eᵢₘₚᵣₒₚₑᵣ | 38.34 ± 0.87 |
| E_{VDW} | 6.59 ± 0.49 |

RMSD of the residue E3-T120 for final 19 structures to REM structure^{a}

| | |
|---|---|
| Backbone (N, Cα, C′) | 0.39 ± 0.09 Å |
| Heavy atoms | 0.97 ± 0.09 Å |

Ramachandran analysis for REM structure^{b}

| | |
|---|---|
| Most favoured region | 84.9% |
| Additionally allowed region | 14.2% |
| Generally allowed region | 0.9% |
| Disallowed region | 0.0% |

^{a}Number of structures used in RMSD calculation was 19.;
^{b}Ramachandran analysis was performed using PROCHECK-NMR program (Laskowski, R. A. et al., J. Biomol. NMR 1996, 8, 477-486).

Mabε's NTD consists of the residues 1-86, forming eight β-strands (FIG. 3B). The NTD is connected to CTD via the linker residues R87-D91. The CTD residues 92-100 form helix α1, including amino acids E92-G100, and helix α2 with residues P104-L117, which are very similar to the solution Mtε- (Joon, S. et al., FEBS J. 2018, 285, 1111-1128) and the cryo-EM Msε structure (Guo, H. et al., Nature 2021, 589, 143-147). Both C-terminal helices of Mabε are connected by a short loop (residues S101-D103). Mab's helix 1 and -2 are significantly shorter compared to their bacterial or human counterparts, indicating differences in energy coupling.

Similar to Mtε, the C-terminal amino acids A108, R111, A112, R115 and A116 are oriented to the NTD, forming a domain-domain interface between the NTD and CTD via the NTD residues D47, D48, A49, A50, V51 and W62. A series of interdomain NOEs between the NTD and CTD were observed between those residues from ¹³C/¹⁵N-NOESY-HSQC- and ¹H/¹³C-NOESY-HSQC spectra. Residues G118-V121 of the C-terminal helix α1 do not participate in the interaction between the NTD and CTD but interact with the helix α2 residues L114-L117.

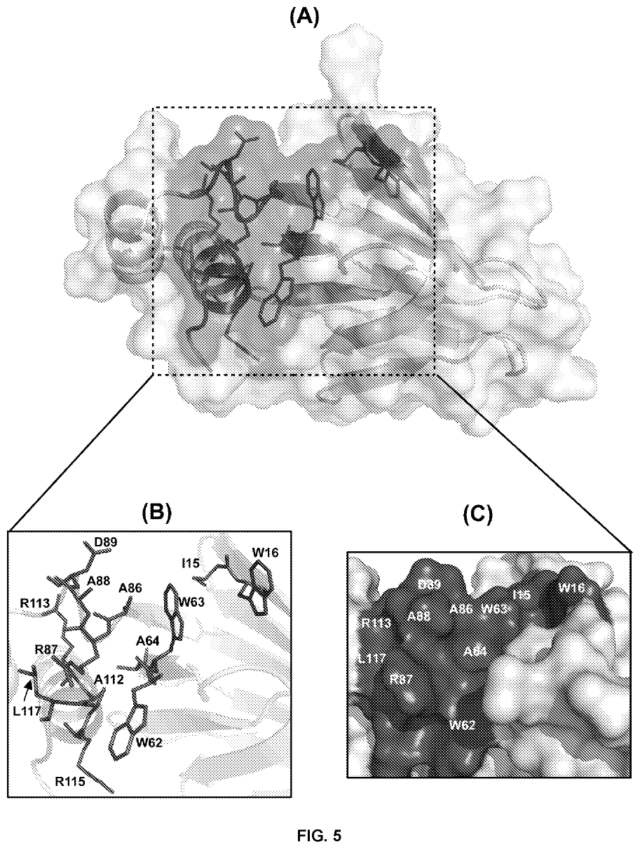

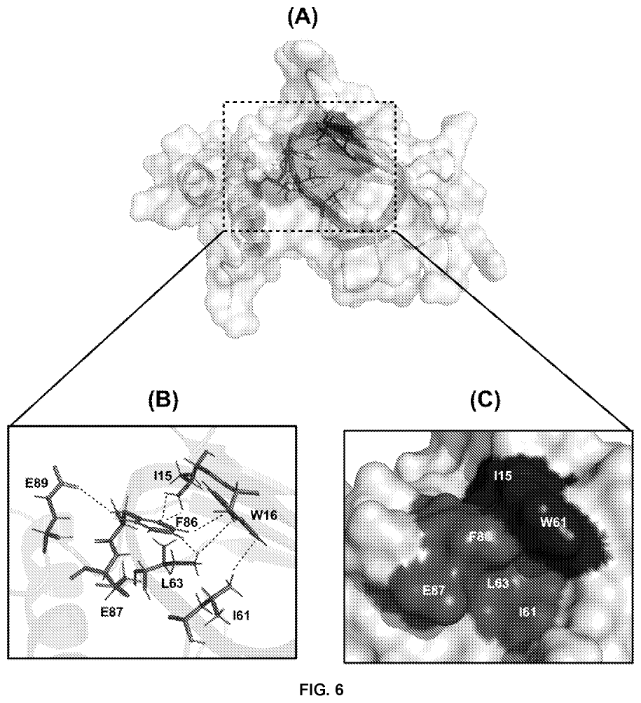

In addition, a number of hydrophobic interactions and hydrogen bonds were observed in NOEs between the N-terminal residues A10-W16 and the epitopes L61-A64 and A86-D89, respectively. Interestingly, 115, W16, L61, W63 and A87 form a hydrophobic cleft (FIGS. 5A-B) as observed in Mtε (FIG. 6). However, the hydrophobic cleft of Mabε shows a different surface shape compared with that of Mtε. In addition to the mycobacterial conserved W16, Mabε contains two additional tryptophan residues (W62 and W63; FIG. 3B) around the hydrophobic pocket instead of R62 and 163 in Mtε, with W63 and W16 forming part of the hydrophobic cleft (FIG. 5C). Furthermore, a structural comparison of Mab γ-ε interface residues with its Mtb counterpart revealed variations at the β1/β2 loop (A10-E14) and β8/β9 loop (A81-S83) segments, which are involved in key interaction with the Mab γ subunit (see below Examples). Additionally, the linker loop segment (α₁/α₂) connecting the NTD with the C-terminal helices of Mabε with varied residues (A86-D89, V106, Q109) also influences interdomain contacts. Taken together, the hydrophobic cleft comprising the tryptophan residues, γ-ε interface motif residues and Mabε's C-terminal residues V106 and Q109 could serve as selective Mabε epitopes for target-based drug discovery efforts.

Taken together, we have determined the first atomic solution structure of Mab's F-ATP synthase subunit ε. The structural details provide insight into critical- and Mab specific epitopes relevant in coupling proton-translocation and the final synthesis of the energy of life, ATP, within the energy converter F-ATP synthase of the pathogen.

### Example 3. Dynamic Features of Mabε

### NMR Relaxation Measurements of Mabε

In order to study the dynamic behaviour of Mabε, ¹⁵N relaxation NMR experiments were performed at 298 K of uniformly ¹⁵N-labeled Mabε (0.3 and 0.5 mM) on a 700-MHz NMR spectrometer. Amide ¹⁵N relaxation data of R1, R2 and heteronuclear ¹H-¹⁵N-NOE were recorded as described (Joon, S. et al., FEBS J. 2018, 285, 1111-1128). R1 data were measured with eight different relaxation delays of 5, 65, 145, 246, 366, 527, 757 and 1148 ms, and R2 data were obtained using seven different relaxation delays of 17, 34, 51, 85, 102, 119 and 136 ms. Duplicated time points (246 ms for T1, and 17 and 51 ms for T2) were used for estimation of the error. Steady-state heteronuclear ¹H-¹⁵N-NOE spectra were recorded with and without 5 s of ¹H proton saturation. The relaxation rates and error estimation were determined using SPARKY, and the relaxation data were analysed and fitted to model-free equation using TENSOR2 (Dosset, P. et al., J. Biomol. NMR 2000, 16, 23-28).

### Results and Discussion

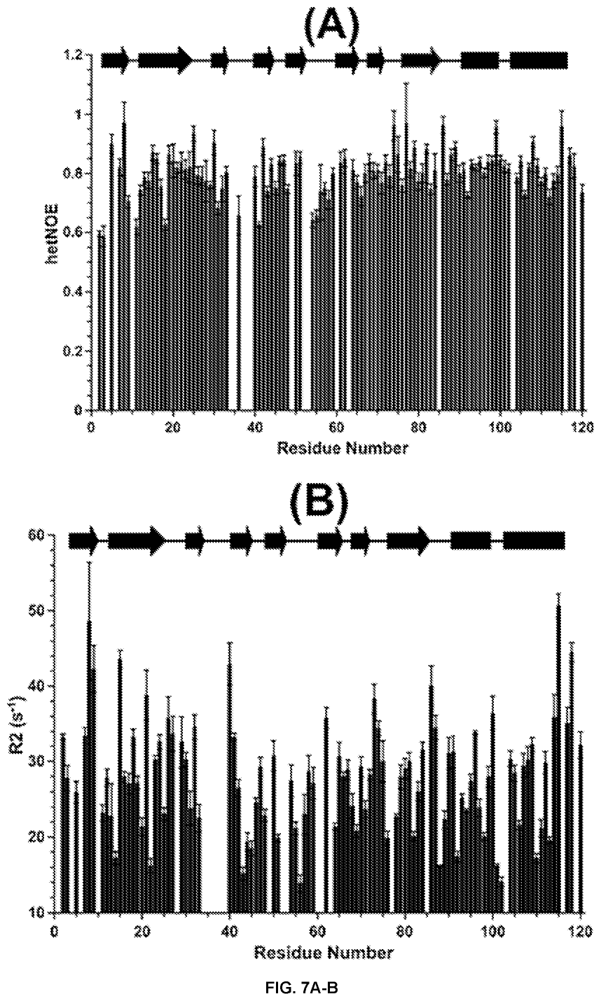

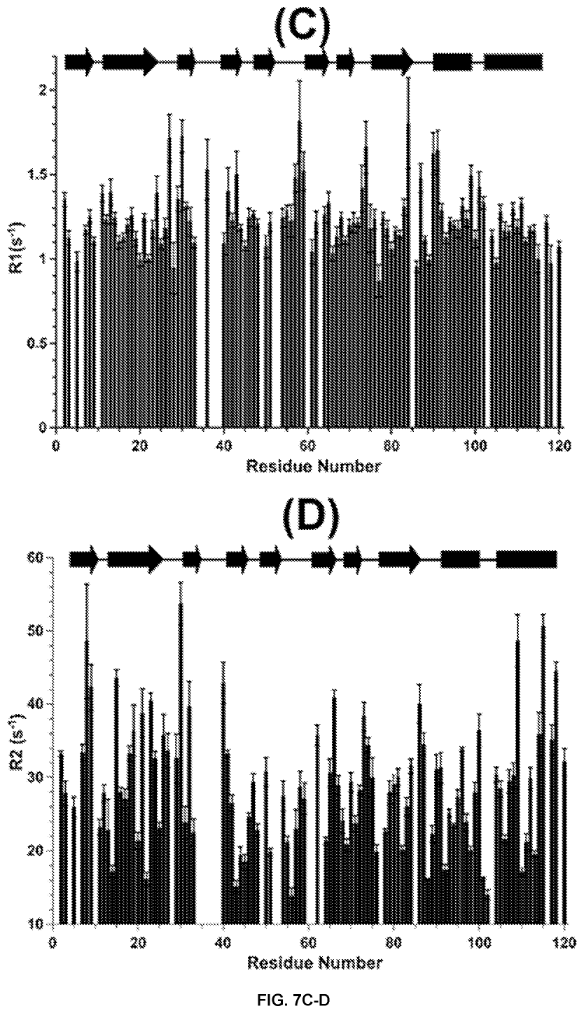

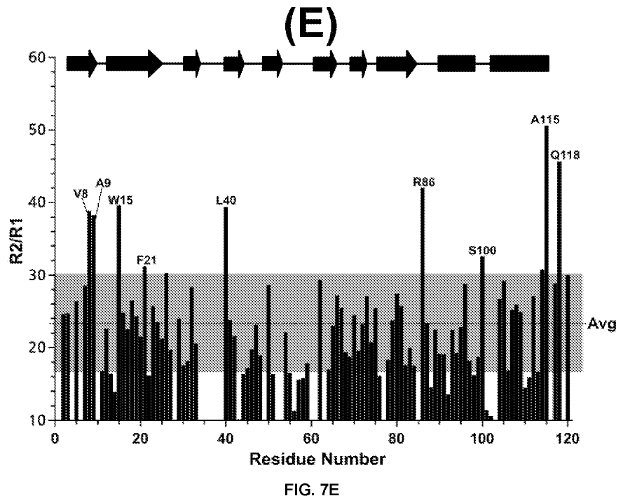

To study the internal motion and the overall domain dynamics, ¹⁵N relaxation data of MabE were measured (FIGS. 7A-E). Among the 117 nonproline residues of Mabε, 97 residues were selected for the dynamics data analysis except 20 residues, which did not appear in the ¹H-¹⁵N HSQC spectra or could not be measured due to extremely low signal-to-noise ratio sensitivity during NMR measurements.

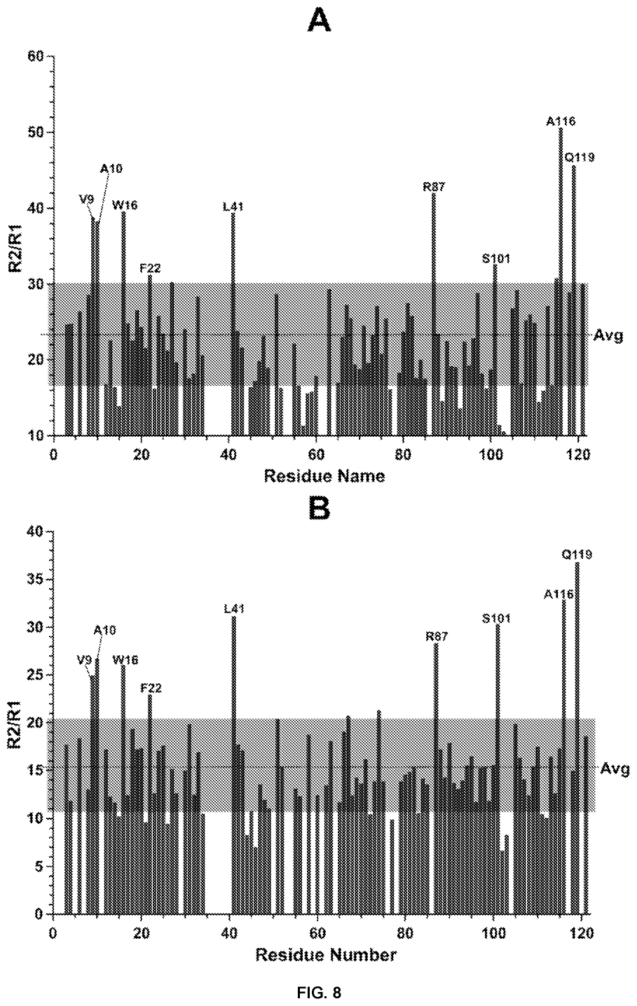

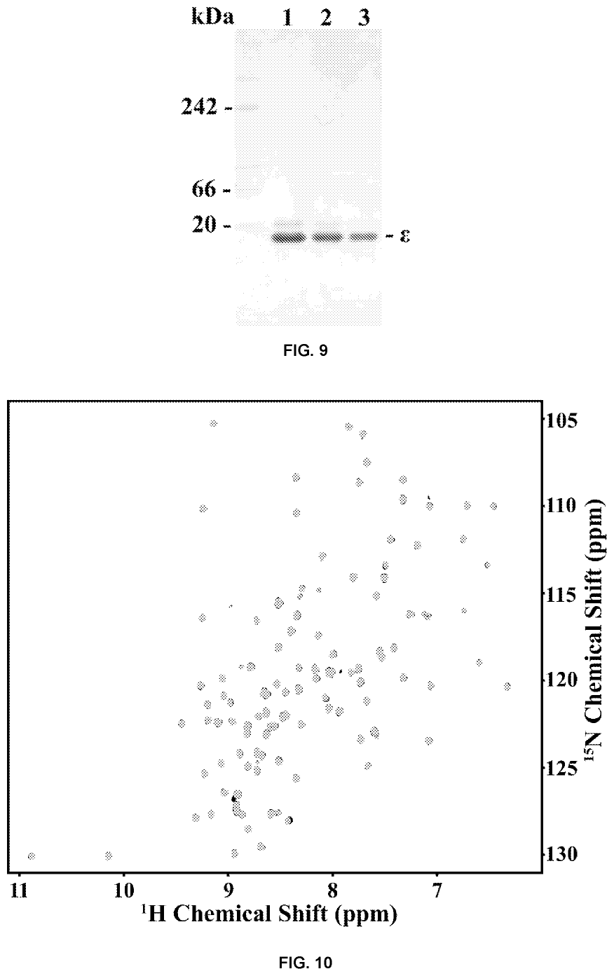

To identify any concentration effect, we performed relaxation measurements at a MabE concentration of 0.3 and 0.5 mM, respectively. The average values of the R2/R1 ratio increased from about 16 (at 0.3 mM of MabE) to about 23 (at 0.5 mM of MabE), indicating that the R1 and R2 values could be slightly affected due to a concentration-dependent monomer-to-dimer equilibrium of Mabε, especially regarding the R2 values (FIGS. 8A-B). A Native PAGE (FIG. 9) revealed Mabε to be mostly monomeric (90-95%) with a minor amount of dimer formation, which was concentration-dependent (0.6 mM, 0.15 mM and 0.07 mM). Importantly, the residues showing relatively high R2/R1 values, such as V9, A10, W16, F22, L41, R87, S101, A116 and Q119, have a similar distribution of R2/R1 values, highlighting that the protein concentration had no critical effect to the dynamic motion of residues in Mabε (FIG. 8B). The average tumbling correlation times (zₑ) for Mabε are about 11.6 ns (at 0.5 mM concentration) and 9.4 ns (at 0.3 mM of concentration). The zₑ value at 0.3 mM of Mabε is similar to the estimated value of τ_{c}≈9.2 ns calculated from the HYDRONMR software (de la Torre, J. G., Huertas, M. L. & Carrasco, B., J. Magn. Reson. 2000, 147, 138-146), underlining that the measured values at 0.3 mM of Mab reflect the real dynamic behaviour of the protein in solution. Based on these results, we continued our structure determination using the experimental data collected at a protein concentration of 0.3 mM. As shown in FIG. 7A, most of the residues adopting a regular secondary structure show heteronuclear NOE values close to the theoretical maximum (0.8-1.0). The average heteronuclear NOE value of Mabε is about 0.82, and the profile of heteronuclear NOEs reveals that the secondary structural regions are relatively rigid. The calculated order parameters (S²) were in the range of 0.7-1.0. Relatively small values of order parameters (<0.6) were only found for the residues in the hinge region between α1 and α2 helices, including amino acids G100 and E102, respectively. About 95% of the residues in Mabε have order parameter S² values >0.7, indicating that the protein in general is rigid (FIG. 7B). The analysis of ¹⁵N R2/R1 ratios reflects the strong correlation between the NTD and CTD (FIGS. 7C-E). The values of the R2/R1 ratio at a concentration of 0.3 mM distributed from 10 to 20 for most of the residues except for residues V9, A10, W16, F22, L41, R87, S101, A116 and Q119 (FIG. 7E). Residues with R2/R1 values being higher than standard deviation (FIG. 7E) include amino acids V9, A10, W16, F22 and L41, the linker residue R87, and the C-terminal residues S101, A116 and Q119. Amino acid W16 shows a significant increase in the R2/R1 value. Importantly, residues R87, S101, A116 and Q119 are related to NTD-CTD interactions or interhelical contact between α1 and α2. ¹⁵N relaxation data reflect that dynamic motion of these residues strongly correlated to the interaction and communication between the NTD and CTD, which may indicate conformational rearrangements of Mabε during coupling. By contrast, the residues F22 and L41 are not directly involved in interdomain interaction. However, it can be proposed that these residues might be related to intersubunit interaction inside the F-ATP synthase. In conclusion, the ¹⁵N relaxation data support the interdomain interactions between the NTD and CTD and provide information about key residues related to the enzymatic mechanism of coupling proton translocation in the F₀ domain and ATP formation in the catalytic F₁ headpiece.

Thus, here, we present the atomic structure of Mab's subunit E (MabE) derived from NMR solution data, and a dynamic characterization of the protein in solution. The dynamic characterization sheds light into the interdomain interactions between the NTD and CTD, and critical amino acids within the subunit for coupling processes within this engine.

### Example 4. Mabε does not Bind ATP

### Whole-Cell ATP Measurement

Clear 96-well flat-bottom cell culture plates (Nunc) were filled with 100 μL of CAMH medium in each well. The compound was added to the first well in each row to create two times the desired highest final concentration. Subsequently, a 16-point twofold serial dilution was carried out starting from the first well in each row. Mab, which was grown to mid-exponential phase, was diluted to an optical density at 600 nm (OD₆₀₀) of 0.1; 100 μL of the diluted culture was added to each well to create a final OD₆₀₀ of 0.05 in all the wells. The plates were incubated at 37° C. on a standing incubator for 24 h. At the end of the incubation period, the samples were measured for their intrabacterial ATP content by employing the BacTiter-Glo microbial cell viability assay (Promega), which was carried out according to the manufacturer's instructions as described previously (Hotra, A. et al., Angew. Chem. Int. Ed. Engl. 2020, 59, 13295-13304). Fifty microliters of each sample was mixed with 50 μL of the BacTiter-Glo reagent in each well of an opaque, white, 96-well, flat-bottom Nunc plate. Luminescence was measured with Cytation 5 multi-mode reader after 10 min of incubation of the plate in the dark at room temperature. The background luminescence reading was subtracted from the luminescence readings of all the samples. The ATP amount is directly proportional to the relative luminescence units. The graph of the results was made using the GRAPHPAD PRISM 8 software (GraphPad Prism 8 Software Inc., San Diego, CA, USA).

### Results and Discussion

In thermophilic Bacillus PS3, Bacillus subtilis and chloroplasts, the C terminus of E is described as a mobile regulatory element, altering its conformation in response to nucleotide conditions or the ion motive force (IMF). ATP binding in E of thermophilic Bacillus PS3 and Bacillus subtilis forces the C-terminal helices into a hairpin conformation, which extends in the absence of the nucleotide, leading to an inhibited ATP hydrolysis state. Here, we used the high-quality NMR spectra of Mabε and performed NMR titration experiments in the presence of MgATP (molar ratio of Mabε to MgATP of 1:10). As demonstrated in the ¹⁵N HSQC spectrum presented in FIG. 10, no obvious chemical shift was observed after the addition of the nucleotide, reflecting that Mabε does not bind ATP. ATP binding of the M. smegmatis subunit E was also not observed in the recent cryo-EM structure of the M. smegmatis F-ATP synthase (Guo, H. et al., Nature 2021, 589, 143-147), underlining that the mycobacterial E subunits are not regulated via nucleotide binding to this rotary subunit.

### Example 5. Receptor-Ligand-Based Pharmacophore Model

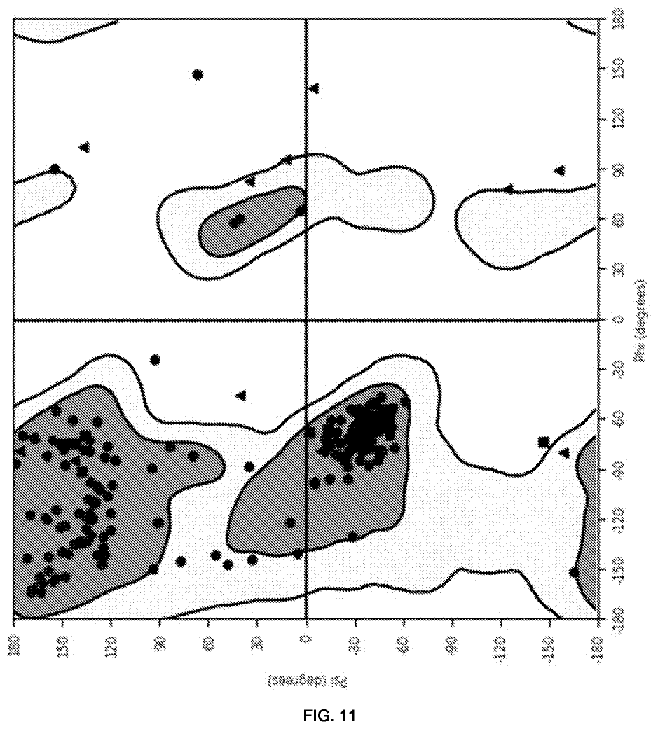

Generating a Mab Subunit γ Homology Model Firstly, a Mab subunit γ (Mabγ) model was generated from its closest homolog M. smegmatis subunit γ (pdb 7JG5, Guo, H. et al., Nature 2021, 589, 143-147) as template using prime tools (Schrödinger release (2020-4) prime. New York, NY:Schrödinger, LLC; 2019; Jacobson, M. P. et al., Proteins 2004, 55, 351-367; and Jacobson, M. P. et al., J. Mol. Biol. 2002, 320, 597-608). The quality of the model was analysed using the Ramachandran plot. Only 10 loop residues of subunit γ, which lacked coordinates from the template, were seen in disallowed regions. The rest of the protein structural elements were in favourable/allowed regions (FIG. 11). Next, the assembly of the Mabγ model and our Mabε NMR structure (PDB: 7VIL) was done by superimposing both to subunits γ and to ε of the cryo-EM M. smegmatis F-ATP synthase (pdb 7JG5, Guo, H. et al., Nature 2021, 589, 143-147), which highlight the Mab γA42-A56 interaction site with Mabε as site point to dock the ligands.

The Mabε solution structure was prepared by adding any missing hydrogens at pH 7.0, by correcting bond orders and energy minimization until the heavy atoms are converged to 0.3 Å using the OPLS3e force field in Protein preparation tool of maestro Schrödinger suite of programs (Harder, E. et al., J. Chem. Theory Comput. 2016, 12, 281-296; and Schrödinger release (2020-4) OPLS3e. New York, NY:Schrödinger, LLC; 2019). The refined structure was utilized for structure-based virtual screening studies.

### 3D Ligand Database Preparation

ChemDiv vendor library was employed using the default settings in Phase ligand preparation (Schrödinger release (2020-4) phase. New York, NY: Schrödinger, LLC; 2019; and Dixon, S. L. et al., J. Comput. Aided Mol. Des. 2006, 20, 647-671) and by checking skip reactive functional groups in ligand filtering options. ADMET properties were calculated separately on a focused library, obtained from a pharmacophore database search, using Qikprop tool (Schrödinger release (2020-4) QikProp. New York, NY: Schrödinger, LLC; 2019).

### Pharmacophore Modelling

Superimposition of the solution NMR Mabε structure to the cryo-EM M. smegmatis F-ATP synthase structure (pdb 7JG5, Guo, H. et al., Nature 2021, 589, 143-147) suggested the crucial rotary γ subunit segment involved in protein-protein interaction with subunit E within the central stalk domain. The coordinates of the Mab cA42-A56 stretch of the subunit γ segment interactions with Mabε were used to define site points to develop a receptor-ligand pharmacophore model using Phase tools of maestro Schrödinger suite of programs (Schrödinger release (2020-4) phase. New York, NY: Schrödinger, LLC; 2019; and Dixon, S. L. et al., J. Comput. Aided Mol. Des. 2006, 20, 647-671).

### Docking and Scoring

Database screening with receptor-based pharmacophore resulted in a focused library of about 30,000 compounds, which was used as input for molecular docking using GOLD CSD suite of programs 2020 (Jones, G. et al., J. Mol. Biol. 1997, 267, 727-748). As mentioned earlier, the Mab γA42-A56 stretch was used to site-point to dock the ligands to Mabε. GOLD & CHEMPLP (Jones, G. et al., J. Mol. Biol. 1997, 267, 727-748; and Korb, O., Stutzle, T. & Exner, T. E., J. Chem. Inf. Model. 2009, 49, 84-96) scoring functions were used to score and rank the fitness of ligands to the protein. Ligands with best CHEMPLP fitness scores were assessed for molecular interactions to the residues involved in interaction with the γA42-A56 epitope, leading to a selection of 10 hit molecules for experimental assays.

### Results and Discussion

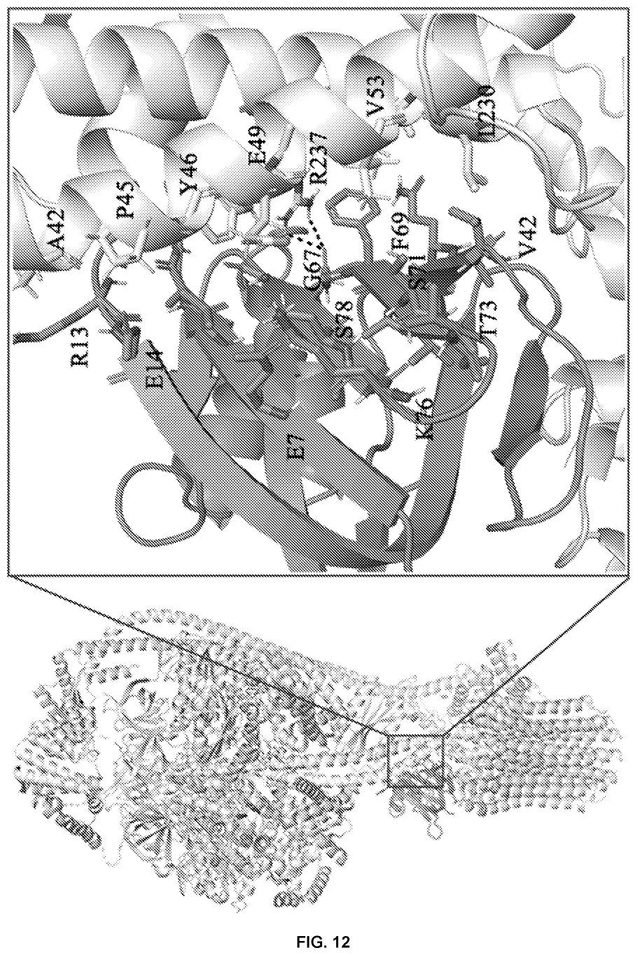

The structural insights of Mabε were used to identify novel compounds targeting this subunit with the aim to disturb interactions between the central rotor subunits γ and ε, and thereby disrupting the process of coupling. With a sequence identity between Mabγ and M. smegmatis γ of 79.4%, a Mabγ homology model with good stereochemical quality could be built. With the exception of the loop residues, which lacked coordinates in the template cryo-EM M. smegmatis F-ATP synthase structure (pdb 7JG5, Guo, H. et al., Nature 2021, 589, 143-147), most of the Mabγ residues were seen in favourable and allowed regions (FIG. 11). Superimposition of Mabγ and Mabε onto the respective subunits of the M. smegmatis F-ATP synthase cryo-EM structure (pdb 7JG5, Guo, H. et al., Nature 2021, 589, 143-147) showed an RMSD of 0.34 and 0.84 Å, respectively, and that a stretch of Mabγ residues A42-A56 and amino acids γL230-L234 forms an interaction interface with amino acids V9 and F69 of Mabγ. Furthermore, γR237 of the neighbouring Mab γ-helix mediates polar contacts with residue G67 Mabε (FIG. 12).

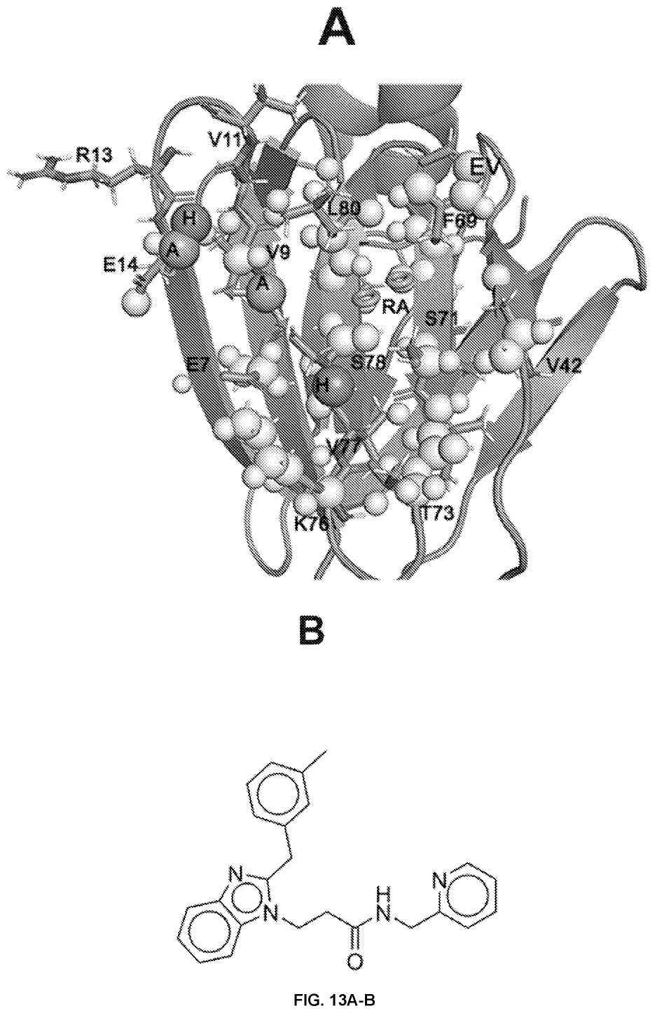

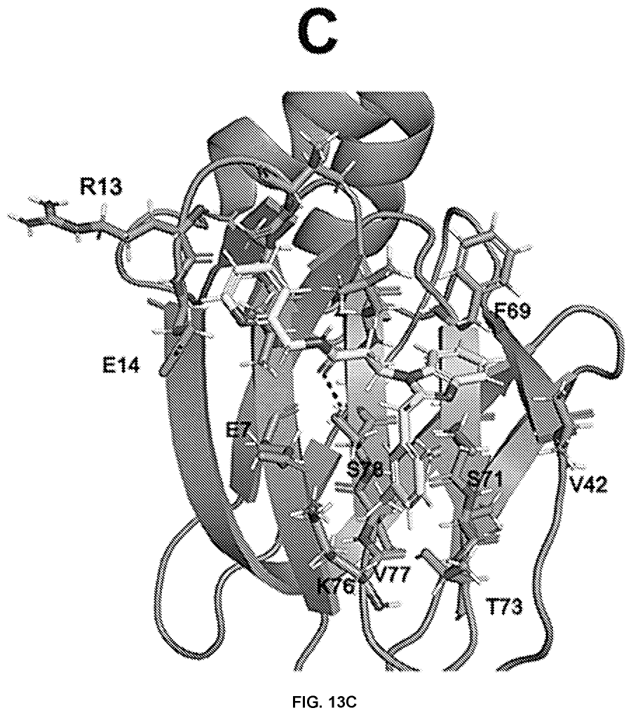

A receptor pharmacophore model was developed on the Mabε residues in the interaction vicinity of the γA42-A56 stretch. As shown in FIG. 13A, a six-feature model was computed, comprising two acceptor (A, labelled spheres), two hydrophobic (H, labelled spheres) and two ring aromatic (labelled as RA) features. The acceptor groups anchor Mabε residues E14 and S78 side-chain atoms, while the two hydrophobic features are positioned in the vicinity of amino acids V9, V11, V77 and L80, and methylene (—CH₂—) atoms of S78 and K76 residues, respectively. The ring aromatic features are in close vicinity to amino acids V42, F69 and L80. Database screening with this pharmacophore model yielded about 30,000 molecules from the 1.3 million ChemDiv commercial library (San Diego, CA, USA) for further virtual screening studies. Afterwards, molecular docking (Jones, G. et al., J. Mol. Biol. 1997, 267, 727-748) studies were carried out using GOLD (Jones, G. et al., J. Mol. Biol. 1997, 267, 727-748)/CHEMPLP (Korb, O., Stutzle, T. & Exner, T. E., J. Chem. Inf. Model. 2009, 49, 84-96; Gehlhaar, D. K., Bouzida, D. & Rejto, P. A., Reduced dimensionality in ligand-protein structure prediction: covalent inhibitors of serine proteases and Design of Site-Directed Combinatorial Libraries. In: Parrill A L, Reddy M R, editors. Rational drug design. Washington, DC: American Chemical Society; 1999. p. 292-311; and Gehlhaar, D. K. et al., Chem Biol. 1995, 2, 317-324) scoring functions to rank the best poses. Further ligands with unfavourable ADMET (absorption, distribution, metabolism, excretion and toxicity) properties were removed using the QIKPROP tool (Schrödinger release (2020-4) QikProp. New York, NY: Schrödinger, LLC; 2019). These virtual screening steps have led to a selection of 10 molecules for experimental evaluation.

### Example 6. Ep1MabF1, a Novel Mab FATP Synthase Inhibitor Targeting Subunit ε

The insights in Examples 2 and 3 were used for a pharmacophore modelling and compound screening, resulting in the hit molecule Ep1MabF1, which was tested for growth- and whole-cell synthesis inhibitory activity. ATP Docking studies were performed by following the protocol in Example 5.

### Antimycobacterial Activity and Minimum Inhibitory Concentration Determination

The M. abscessus subsp. abscessus ATCC 19977 strain was used. The Mab strain was maintained in CAMH broth, which was prepared according to the manufacturer's instructions. The growth inhibition dose-response assay was carried out using the broth microdilution method as described previously (Moreira, W., Aziz, D. B. & Dick T., Front. Microbiol. 2016, 7, 199). The MIC₅₀ reported represents the concentration that inhibits 50% of growth compared with the untreated culture.

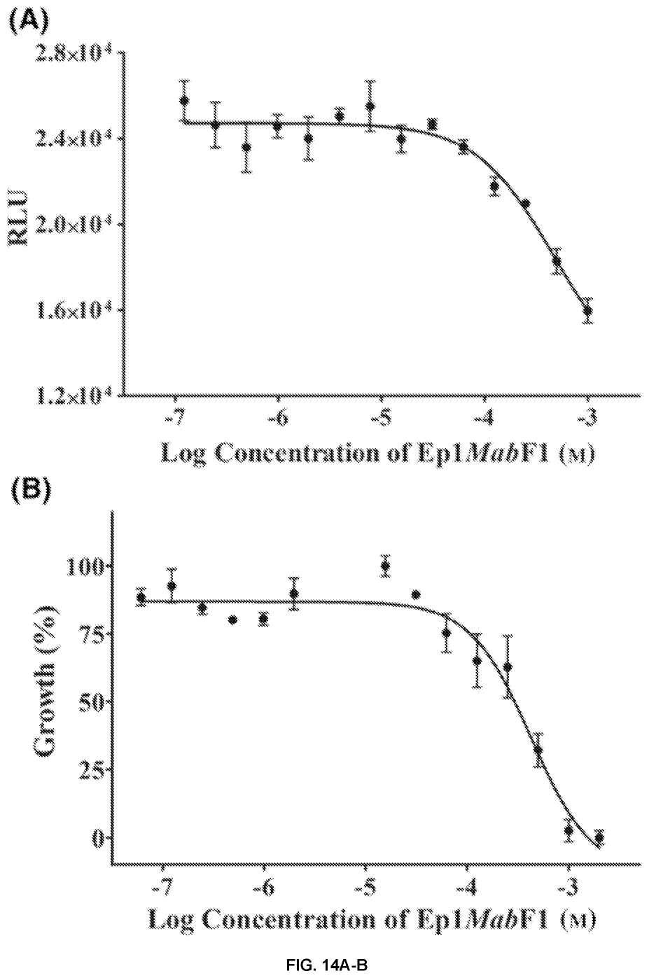

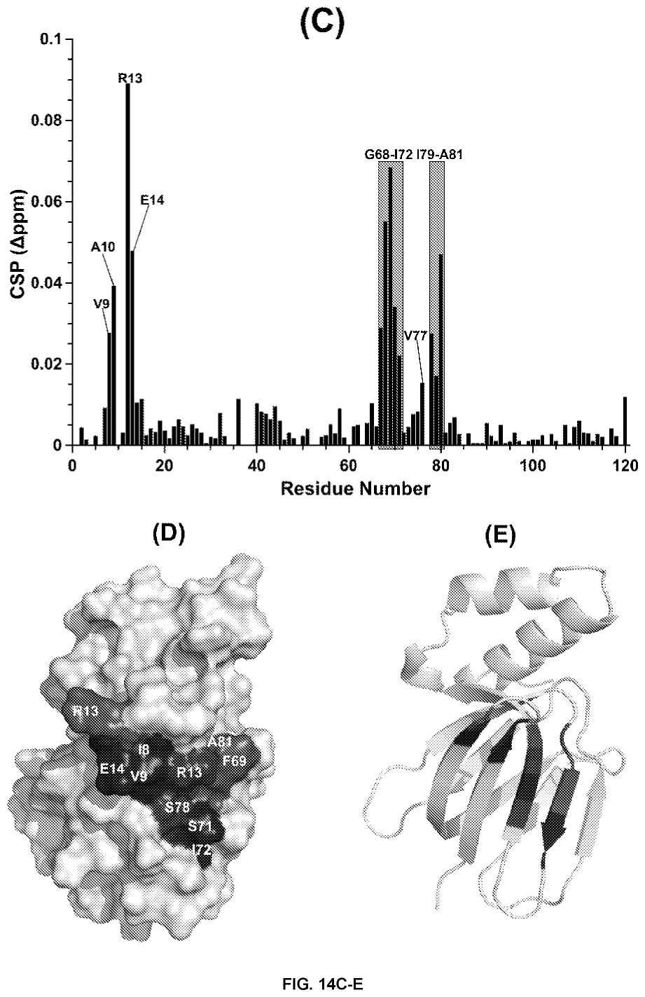

Results and Discussion Among the 10 molecules (described in Example 5) assayed, one molecule (#1723; 3-(2-(3-methylbenzyl)-1H-benzo[d]imidazol-1-yl)-N-(pyridin-2-ylmethyl)propanamide (FIGS. 13B-C), named Ep1MabF1) was shown to inhibit the intracellular ATP levels at moderate potency with an inhibitory concentration (IC₅₀) of 600±30 μM (FIG. 14A). Ep1MabF1 displayed potency of M. abscessus subsp. abscessus growth inhibition with a minimum inhibitory concentration (MIC₅₀) of 420±14 μM (FIG. 14B). The similar IC₅₀- and MIC₅₀ values determined, indicate that ATP synthesis catalyzed by the F-ATP synthase is the major cause of cell growth inhibition.

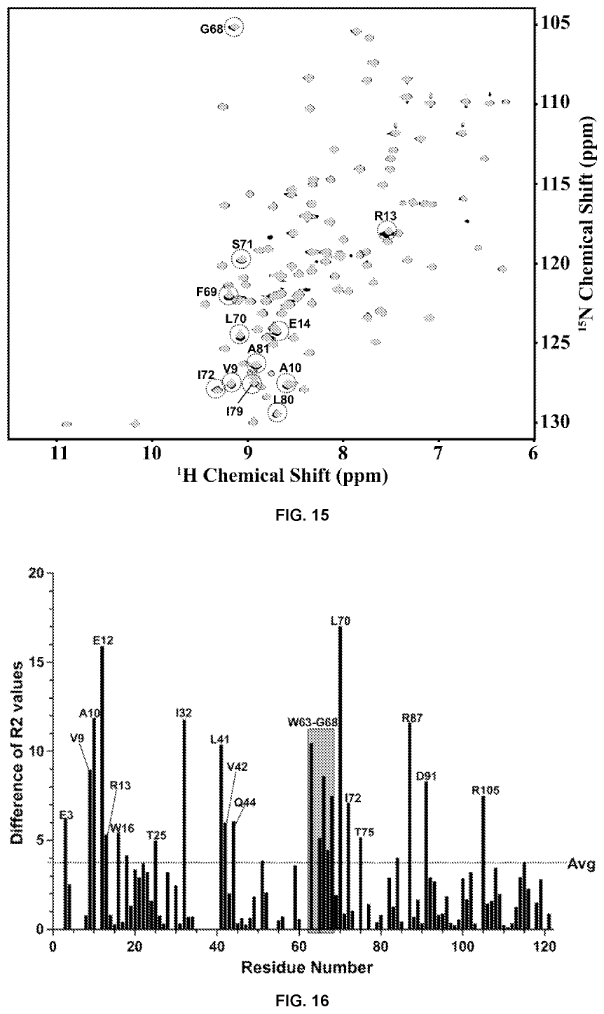

To confirm that Mabε is Ep1MabF1's target, a ¹H-¹⁵N-HSQC NMR titration experiment in the presence (1:5 molar ratio of Mabε and Ep1MabF1) and absence of the compound was performed. Significant changes in chemical shift were (CSP >0.15 ppm) detected for the backbone resonances of amino acids V9, A10, R13, E14, G68-172, V77 and 179-A81 (FIGS. 14C and 15). Residues showing significant changes of CSP are located and clustered on the β-1, β-2, β-6 and β-7 strands of NTD, indicating that these residues might be directly involved in the interaction with Ep1MabF1 (FIGS. 14D-E). No significant changes were observed in the CTD and other regions of the NTD. In addition, we measured R2 relaxation data in the presence of Ep1MabF1 and calculated the changes of R2 values between the compound-free and compound-bound protein. As shown in FIG. 12, significant increases of R2 values in the presence of Ep1MabF1 were observed for the residues V9, A10, E12, R13, W63-G68, L70, 172 and T75, which are located around the potential binding site. In addition, residues R87, D91 and R105 also showed increased R2 values when compared to the average ones. These residues are distributed in the hinge region of the NTD-CTD (R87 and D91) and the starting region of helix α2 (R105), which is nearby the α1-α2 connection loop, suggesting that an alteration of local structural and dynamical properties of these residues was caused by Ep1MabF1 binding (FIG. 16).

Docking studies revealed that Mabε binds Ep1MabF1 with a good CHEMPLP fitness of 52.19. The main scaffold 1H-benzo[d]imidazole was positioned at the hydrophobic groove encompassing amino acids F69, L80 and V42, and mediates the alkyl-aromatic interactions with these residues. The 3-methylbenzyl fragment on the second position of benzimidazole (main scaffold) was also engaged in hydrophobic interactions with methylene atoms of residues K76 and S78. The propionamide atoms, which link the benzimidazole to the 2-pyridyl group, were in H-bonding interaction with S78. The 2-pyridyl group was positioned into the hydrophobic groove lined by V11, V9 and L80 residues (FIG. 13C). The docking data confirm the observed chemical shift perturbations of the N-terminal, G68-172 and 179-A81 stretches in the ¹⁵N-HSQC NMR titration experiments.

Taken together, H-bonding interactions and hydrophobic interactions would stabilize the ligand binding at this segment and potentially inhibit coupling via the central stalk and finally ATP synthesis inside the catalytic α₃:β₃ headpiece. Further, the NMR and docking experiments confirmed Mabε as a target for Ep1MabF1 and present a novel compound that binds to Mabε and interrupts proper interaction with the rotary γ subunit. The potency of Ep1MabF1 in ATP synthesis inhibition supports the mechanistic importance of the Mabε in coupling, and form a platform for further structure-activity relationship studies (SAR). The successful targeting of Mabε demonstrates the potential to advance this subunit as a new area for the development of anti-Mab F-ATP synthase inhibitors. As such, the first identification of Mab's F-ATP synthase subunit ε as an anti-Mab target and Ep1MabF1 as a novel inhibitor will add to the poor anti-Mab drug pipeline and to the need for novel targets fighting Mab-caused infectious diseases.

1. A compound of formula I:
X-L-Y—Z I
where:
X is selected from phenyl, pyridyl, quinazolinyl, or naphtyl, where the phenyl, pyridyl, quinazolinyl, or naphtyl group is unsubstituted or substituted by one or more of the group selected from Cl, NH₂, pyrrolyl, imidazolyl, tetrazolyl, CH₂NHCONH₂, and CH₂NHSO₂NH₂;
Y is a heteroaryl ring system selected from benzimidazolyl, benzothiazolyl, benzofuranyl, quinazolinyl, and naphthyl, which groups are unsubstituted or substituted by one or more substituents selected from the group consisting of OMe, —O—CH₂—O—, and —O(CH₂)₂O—, where the oxygen atoms in the latter two groups are attached to different atoms on the heteroaryl ring system to form a further ring;
Z is selected from H, phenyl and pyridyl, where the phenyl and pyridyl groups are unsubstituted or substituted by one or more of the group selected from methyl, piperidinyl, benzyl, benzyl-4-OMe, benzyl-4-OCF₃, and benzyl-4-OSF₅;
L is selected from —CH₂NHCOCH₂CH₂— or —CH₂-phenyl-CH₂—, —CH₂—, —NH-pyrrolyl, imidazolyl, and thiazolyl.
or a pharmaceutically acceptable salt or solvate thereof.

2. The compound, or pharmaceutically acceptable salt or solvate thereof, according to claim 1, wherein when X is a phenyl, pyridyl, quinazolinyl, or naphtyl group it is unsubstituted or is substituted by one of a first group of substituents and by one of a second group of substituents, where
the first group of substituents is H or Cl; and
the second group of substituents is NH₂, pyrrolyl, imidazolyl, tetrazolyl, CH₂NHCONH₂, and CH₂NHSO₂NH₂.

3. The compound, or pharmaceutically acceptable salt or solvate thereof, according to claim 1, wherein X is phenyl or pyridyl, optionally wherein X is pyridyl.

4. The compound, or pharmaceutically acceptable salt or solvate thereof, according to claim 1, wherein Y is benzimidazolyl or 5,6-dimethoxy benzimidazolyl.

5. The compound, or pharmaceutically acceptable salt or solvate thereof, according to claim 1, wherein L is —CH₂NHCOCH₂CH₂—.

6. The compound, or pharmaceutically acceptable salt or solvate thereof, according to claim 1, wherein the compound of formula I, or pharmaceutically acceptable salt or solvate thereof, is

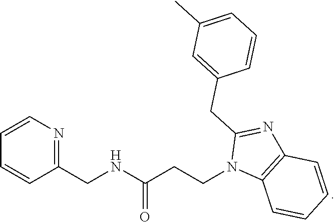 [SMILES: Cc1cccc(Cc2nc3ccccc3n2CCC(=O)NCc2ccccn2)c1]

7. (canceled)

8. (canceled)

9. A method of treatment of a bacterial infection caused by Mycobacterium abscessus, which method comprises the administration of a pharmaceutically effective amount of a compound of formula I as defined in claim 1 or a salt or a solvate thereof, to a patient in need of such treatment.

10. (canceled)

11. (canceled)

12. A method of treatment of a bacterial infection caused by Mycobacterium abscessus, which method comprises the administration of a pharmaceutically effective amount of a compound of formula I as defined in claim 1 or a salt or a solvate thereof, and another therapeutic agent, or a salt or solvate thereof, to a patient in need of such treatment.

13. A method of identifying compounds that can bind to Mycobacterium abscessus F-ATP synthase subunit epsilon, comprising the steps of:
A) electronically screening stored spatial coordinates of a set of candidate compounds against the spatial coordinates comprising;
i) C-terminal amino acid positions A107, R110, A111, R114 and A115 of the Mycobacterium abscessus F-ATP synthase subunit ε, which form a domain-domain interface, or binding pocket, with the N-terminal amino acid residues D46, D47, A48, A49, V50 and W61 of the M. abscessus F-ATP synthase subunit ε, and
ii) amino acid positions γA42-A56 of subunit ε, which forms a protein-protein interface with M. abscessus F-ATP synthase subunit γ,
to identify compounds that can bind to said F-ATP synthase subunit ε, wherein the M. abscessus F-ATP synthase subunit ε comprises the amino acid sequence set forth in SEQ ID NO: 1 and wherein the M. abscessus F-ATP synthase subunit γ comprises the amino acid sequence set forth in SEQ ID NO: 2; and
B) optionally determining whether identified compounds inhibit Mycobacterium abscessus F-ATP synthase subunit epsilon activity.

14. The method of claim 13, wherein a receptor pharmacophore model is developed on the Mycobacterium abscessus F-ATP synthase subunit epsilon residues in the interaction vicinity of amino acid positions A42-A56 of said Mycobacterium abscessus F-ATP synthase subunit γ.

15. The method of claim 13, further comprising molecular docking screening to further rank identified compounds.

16. The method of claim 13, wherein inhibition of Mycobacterium abscessus F-ATP synthase subunit epsilon activity will inhibit F-ATP synthase and M. abscessus growth.

17. The compound, or pharmaceutically acceptable salt or solvate thereof, according to claim 1, wherein X is pyridyl.
